(19) United States
(12) Patent Application Publication
(10) Pub. No.: US 2025/0305049 A1
(43) Pub. Date: Oct. 2, 2025

(54) PURIFICATION OF PLACENTAL SPECIFIC EXTRACELLULAR VESICLES FROM MATERNAL PLASMA TO DETECT PLACENTAL PATHOLOGIES

(71) Applicant: HACKENSACK MERIDIAN HEALTH, INC., Edison, NJ (US)

(72) Inventors: Olivier Loudig, Nutley, NJ (US); Megan I. Mitchell, Nutley, NJ (US); Marwa Khalil, Edison, NJ (US)

(73) Assignee: HACKENSACK MERIDIAN HEALTH, INC., Edison, NJ (US)

(21) Appl. No.: 19/071,303

(22) Filed: Mar. 5, 2025

Related U.S. Application Data
(60) Provisional application No. 63/562,516, filed on Mar. 7, 2024.

(51) Int. Cl. C12Q 1/6883; C12Q 1/6804

(52) U.S. Cl. CPC C12Q 1/6883; C12Q 1/6804; C12Q 2600/158; C12Q 2600/178

(57) ABSTRACT

The present disclosure provides a non-invasive method for early diagnosis of a placental pathology comprising an abnormal formation or arrangement of a placenta in a uterus of a mammalian female subject during pregnancy. Early diagnosis can lead to an improved maternal outcome. The method comprises selectively purifying from plasma of maternal blood a population of small extracellular vesicles (small-EVs) expressing a placenta-specific surface biomarker. The extracellular vesicles comprise micro-RNA cargo. A cargo profile for the small EVs is determined by extracting RNA from the purified population of small EVs. Expression of small non-coding RNAs comprising one or more micro RNAs (miRNAs) encapsulated by the purified population of exosomes is then identified and quantified. The miRNA profile of the placenta specific EVs is then compared to the miRNA profile of a healthy control of the same approximate gestational age.

### CROSS-REFERENCE TO RELATED APPLICATIONS

The present application claims the benefit of priority to U.S. provisional application 63/562,516 (filed Mar. 7, 2024), the entire contents of which are incorporated by reference.

### FIELD OF THE INVENTION

The present disclosure relates to methods for noninvasive early detection of placental pathologies in mammals, including humans.

### BACKGROUND OF THE INVENTION

### Human Placental Development

Human placental development requires coordinated interaction between the trophoblast lineages of the placenta and the maternal endometrium. [Cindrova-Davies, T. and Sferruzzi-Perri, S. Seminars in Cell and Developmental Bio. (2022) 131:63-77]. The human placenta develops from the trophectoderm (TE), the outer layer of the pre-implantation embryo, which forms at ˜5 days post fertilization (dpf). At this stage, the pre-implantation embryo (termed a blastocyst) is segregated into two lineages: the inner cell mass (ICM) and the TE. The polar TE (the part of the TE that is contiguous with the underlying ICM) attaches to the surface epithelium of the uterine mucosa: the endometrium. Although the earliest stages of implantation have not been visualized in humans, morphological observations of early pregnant hysterectomy specimens and higher primates suggest that, following attachment to the uterine surface epithelium at ˜6-7 dpf, the TE fuses to form a primary syncytium. This is the prelacunar phase of placental development. [Turco, M Y and Moffett, A. Development (2019) 146 (22): dev. 163428].

Following implantation, the primary syncytium quickly invades through the surface epithelium into the underlying endometrium, which is transformed during pregnancy into a specialized tissue known as the decidua [Id., citing Schlafke, S. and Enders, AC. Biol. Reprod. (1975) 12:41-65]. By the time of the first missed menstrual period (˜14 dpf), the blastocyst is completely embedded in the decidua and is covered by the surface epithelium (Id., citing Hertig, A T et al. Am. J. Anat. (1956) 98:435-493]. Fluid-filled spaces (lacunae) then appear within the syncytial mass that enlarge and merge, partitioning it into a system of trabeculae. This is the lacunar stage. The syncytium also erodes into decidual glands, allowing secretions to bathe the syncytial mass [Id., citing Hertig, A T et al. Am. J. Anat. (1956) 98:435-493].

The trophoblast cells beneath the syncytium (termed cytotrophoblast cells) are initially not in direct contact with maternal tissue but rapidly proliferate to form projections that push through the primary syncytium to form primary villi (a cytotrophoblast core with an outer layer of syncytiotrophoblast, SCT); this is the villous stage of development. The villous trees are formed by further proliferation and branching, and the lacunae become the intervillous space. Cytotrophoblast cells eventually penetrate through the primary syncytium and merge laterally to surround the conceptus in a continuous cytotrophoblast shell between the villi and the decidua. The blastocyst is now covered by three layers: the inner chorionic plate in contact with the original cavity; the villi separated by the intervillous space; and the cytotrophoblast shell in contact with the decidua. [Turco, M Y and Moffett, A. Development (2019) 146 (22): dev. 163428].

Soon afterwards, around day 17-18, extraembryonic mesenchymal cells penetrate through the villous core to form secondary villi. By day 18 dpf, fetal capillaries appear within the core, marking the development of tertiary villi. The villous tree continues to rapidly enlarge by progressive branching from the chorionic plate to form a system of villous trees. Where the cytotrophoblast shell is in contact with the decidua (the maternal-fetal interface), individual cytotrophoblast cells leave the shell to invade into decidua as extravillous trophoblast (EVT) in a process closely resembling epithelial-mesenchymal transition (EMT). In this way, by the end of the first trimester, the blueprint of the placenta is established. [Turco, M Y and Moffett, A. Development (2019) 146 (22): dev. 163428].

### Animal Models for the Study of Human Placental Development.

There is no perfect experimental model to investigate human placentation. Disorders such as pre-eclampsia in which the primary defect is failure of placentation are found only in humans and possibly in great apes. [Id., citing Carter, A M. Reproduction (2011) 141:391-396].

The differences between mouse and human placentation are considerable. Besides deviations in gross morphology and specific trophoblast cell types, blastocysts implant differently in mice, trophoblast invasion is very shallow and remodeling of uterine arterial vessels largely depends on maternal factors [Knofler, M. et al. Cellular & Molecular Life Sciences (2019) 76:3479-3496, citing Carter, A M. Placenta (2007) 28 (Suppl. A): S41-S47]. Moreover, key regulators of placental development differ between mice and humans [Id., citing Knofler, M. et al. Placenta (2001) 22 (Suppl. A): S83-S92], making the mouse an imperfect model of human placentation.

The laboratory rat, a rodent model where the trophoblast extends deeply into the uterine wall to remodel the artery feeding the placentation site, has been used to study arterial transformation at the maternal-fetal interface [Turco, M Y and Moffett, A. Development (2019) 146 (22): dev. 163428, citing Soares, M J et al. Placenta (2012) 33:233-243].

Abnormal placentation in the first trimester is considered as an underlying cause of various pregnancy complications such as miscarriage, unexplained stillbirth, pre-term labor placental abruption, pre-eclampsia and intrauterine growth restriction. [Knofler, M. et al. Cellular & Molecular Life Sciences (2019) 76:3479-3496, citing Brosens, J J et al. Am. J. Obstet. Gynecol. (2002) 187:1416-1423; Fisher, S J. Am. J. Obstet. Gynecol. (2015) 213: S115-S122; Hustin, J. et al. Placenta (1990) 11:477-486; Khong, T Y et al. Br. J. Obstat. Gynaecol. (1986) 93:1049-1059; Khong, T Y et al. (1987) Br. J. Obstet. Gynecol. (1991) 98:648-655; Pijienborg, R. et al. Br. J. Obstet. Gynecol. (1991) 98:648-655; Romero, R. et al. Best Pract. Res. Clin. Obstet. Gynecol. (2011) 25:313-327]. 7-13].

Placenta accreta spectrum (PAS), also referred to as morbidly adherent placenta, denotes the abnormal adherence and invasion of the placental trophoblast into the uterine myometrium [1-4]. PAS is pathologically classified into three different divisions depending on the depth of trophoblast invasion; placenta accreta which refers to the attachment of the placenta into the myometrium without intervening decidua, placenta increta refers to the invasion of the trophoblast into the myometrium and placenta percreta (the most severe), which refers to the invasion of the trophoblast through the myometrium, serosa and into surrounding structures and tissues [5,1]. Although the pathogenesis of PAS remains unclear, its pathophysiology is commonly correlated to an absence of normal decidua basalis which usually occurs as a result of uterine scarring from previous surgical trauma (e.g., cesarean delivery), which enables the trophoblast to attach and invade into the scarred myometrium [6-8]. The degree of abnormal placentation is correlated with severe clinical implications, as the failure of the placenta to spontaneously detach from the uterus during delivery is associated with significant risk of maternal hemorrhage, which can result in disseminated intravascular coagulation (DIC), multisystem organ failure, and death in extreme cases [9-15].

The incidence of placenta accreta spectrum (PAS) has increased and has been reported to be as high as 1±2 in 1000 in several locations across North America [16-18]. These conditions have become one of the leading causes of postpartum hemorrhage in the U.S. and remain a significant contributor to maternal morbidity and mortality. Although the risk factors of prior uterine damage (primarily from cesarean section) are of major epidemiological importance for identifying possible patient cases, there are nevertheless many cases where the presence of this risk factor is not associated with PAS. In clinical practice, up to 50% of pregnancies with PAS remain undiagnosed until delivery and thus are associated with an increased risk of morbidity [6,7]. Considering that there are currently no clinical diagnostic assays routinely used to detect the development of PAS, new and improved paradigms are urgently needed for the early and accurate diagnosis of this condition. Although both ultrasound and MRI have been used effectively to diagnose certain PAS cases, the subjectivity involved in assessing visual markers remains restricted to expert use and trained professionals [19-22]. The consequence is that many cases of PAS remain undiagnosed or misdiagnosed, and lead to poor maternal outcomes. In view of these circumstances, having biomarkers for the detection of PAS would be of considerable diagnostic benefit, enabling physicians to prepare early enough for the complex delivery often required in these cases [23-27]. Additionally, the identification of the molecular pathways involved in the upregulation of placental tissue invasion might enable the design of novel therapeutic tools to help reduce the degree of invasion and in turn regulate the incidence of these abnormal placental conditions.

To date, many biomarker studies have been conducted but have been inconclusive, and there are still no clinically reliable urine or blood biomarkers for the early detection of PAS. Although several studies suggest that impaired angiogenesis [28-30], abnormal decidualization [31] and trophoblast factors [32,33] contribute to the pathophysiology of PAS, evaluations of maternal serum for angiogenic and ancuploidy markers, as well as fetal circulating DNA, obtained during non-invasive prenatal screening, have not identified robust biomarkers, which could provide a clinically useful diagnostic test for PAS [34-50]. Nevertheless, over the last several years many studies have been focused on the identification of fetal biomarkers circulating in maternal blood due to its direct contact with the placenta. Several placental and fetal hormones routinely used in the screening for aneuploidy have been found to be differentially expressed in the serum of women with PAS compared with those with placenta previa (a condition in which the non-invasive placenta lies low in the uterus and regarded as a risk factor for placenta accreta spectrum) [44, 45]. Recently, there has been increasing interest in the role of cell-free fetal DNA (cffDNA) for screening and diagnosis of PAS, but these studies are still ongoing [37,38].

Placental cells have been shown to shed extracellular vesicles (EVs), both in fetal and maternal circulations, which can be detected as early as the 6th week of pregnancy [51,52]. The abundance of these circulating placental EVs detectable in maternal blood increases in amount throughout the duration of pregnancy with maximal levels being detected at term [53]. As such these circulating placental EVs represent a potentially valuable source of placenta-specific biomarkers for the non-invasive diagnosis of PAS [54]. One of the defining characteristics of placental EVs is the presence of placental alkaline phosphatase (PLAP), a fetal protein unique to the placenta, on their surface [55-57]. While other alkaline phosphatases can be found in other tissues and are therefore not specific to the placenta, PLAP lacks the last 24 amino acids in its N-terminal region, thereby making it specific to the placenta, thereby providing unique epitopes for antibody capture of circulating placental EVs [58]. This modification of PLAP, increases its' substrate specificity as well as its' stability to heat and resistance to chemical inactivation [58]. Its main functions described so far are assistance in the transfer of immunoglobulin G (IgG) from a mother to the fetus and stimulation of fibroblast DNA synthesis and proliferation [58]. As PLAP is known to be a surface protein found in abundance on placental EVs, this unique marker has indeed become popular when assessing placenta-specific EVs [59]. Additionally, several recent studies on placental exosomes have already demonstrated that they can participate in the adaptive immune response in mother and fetus, and that their concentration and function differ in various placental pathologies. [60-63].

Abnormally Invasive Placentation (AIP), also known as Placenta Accreta Spectrum (PAS) is a rare but life-threatening condition in which placental trophoblastic cells abnormally invade the uterus, outer uterine serosa, and in extreme cases tissues beyond the uterine wall. Since there is no clinical assay for the non-invasive detection of AIP, only ultrasound and MRI can be used for its diagnosis. Considering the subjectivity of visual assessment, the detection of AIP necessitates a high degree of expertise and in some instances can lead to its misdiagnosis. In clinical practice, up to 50% of pregnancies with PAS remain undiagnosed until delivery and can be associated with increased risk of morbidity. Although many studies have evaluated the potential of fetal biomarkers circulating in maternal blood, very few studies have evaluated the potential of circulating placental EVs and their miRNA contents for molecular detection of AIP. Thus, to selectively purify placental EVs from maternal blood we customized our robust ultra-sensitive immuno-purification assay, termed EV-CATCHER™, with a monoclonal antibody targeting the transmembrane Placental Alkaline Phosphatase (PLAP) protein, which is unique to the placenta and present on the surface of placental EVs. Then, as a pilot evaluation we compared the miRNA expression profiles of placental EVs purified from the maternal plasma of women diagnosed with placenta previa (controls (n=16); placenta lying low in uterus but not invasive) to those of placental EVs purified from the plasma of women with placenta percreta (cases (n=16); placenta with the highest level of invasiveness). Our analyses reveal that miRNA profiling of placenta specific EVs purified from maternal plasma identified 40 differentially expressed miRNAs when comparing these two placental pathologies. Additionally, preliminary miRNA pathway enrichment and gene ontology analysis of the top 14 upregulated and top 9 downregulated miRNAs in placental-EVs, purified from the plasma of women diagnosed with placenta percreta versus those diagnosed with placenta previa, suggests a potential role in control of cellular invasion and motility, which will require further evaluation.

### SUMMARY OF THE INVENTION

According to one aspect, the present disclosure provides a non-invasive method for early diagnosis of a placental pathology comprising an abnormal formation or arrangement of a placenta in a uterus of a mammalian female subject during pregnancy, the method comprising selectively purifying from plasma of maternal blood a population of small extracellular vesicles (small-EVs) expressing a placenta-specific surface biomarker; wherein the extracellular vesicles comprise micro-RNA cargo; determining a cargo profile for the small EVs by extracting RNA from the purified population of small EVs; identifying and quantifying expression of small non-coding RNAs comprising one or more micro RNAs (miRNAs) encapsulated by the purified population of exosomes; and comparing the miRNA profile of the placenta specific EVs to the miRNA profile of a healthy control of the same approximate gestational age; wherein the early diagnosis can lead to an improved maternal outcome.

According to some embodiments of the method, the placental pathology includes placenta previa and placenta accrete spectrum (PAS).

According to some embodiments, the placenta accrete spectrum (PAS) comprises placenta accreta, placenta increta, and placenta percreta.

According to some embodiments, the method comprises an initial ultrafiltration step, an ultracentrifugation step or both to provide a pooled heterogeneous population of biological particles.

According to some embodiments, the purified population of small-EVs is at least 50% pure, at least 55% pure, at least 60% pure, at least 65% pure, at least 70% pure, at least 75% pure, at least 80% pure, at least 85% pure, at least 90% pure, at least 91%, 92%, 93%, 94%, 95%, 96%, 97%, 98% 99% pure or 100% pure.

According to some embodiments, the purified population of small-EVs is homogeneous.

According to some embodiments, the selective purifying is by antibody capture of the placental EVs in the maternal plasma. According to some embodiments, the antibody is a monoclonal antibody raised against a recombinant human PLAP and the placenta-specific biomarker comprises transmembrane placental alkaline phosphatase (PLAP) protein.

According to some embodiments, the monoclonal antibody raised against the recombinant human PLAP is activated with a dibenzocyclo-octyl (DBCO)-ester; the DBCO-modified antibody is coupled to a DNA linker by click chemistry, the antibody-DNA linker conjugates are bound to streptavidin coated well plates pretreated with RNAse A; the purified population of placenta-specific small EVs are released from the streptavidin-coated well plates enzymatically by uracil glycosylase; and the purified population of placenta-specific small-EVs is eluted from the monoclonal PLAP antibody complex by contacting the complex with free PLAP.

According to some embodiments, the method differentiates between small EVs of human women with the placental pathology placenta previa and human women with the placental pathology placenta percreta.

According to some embodiments, the method identifies 40 differentially expressed miRNAs, including miR-21 and, miR-191 and miR-223 with increased expression and miR-451 and miR-486 with decreased expression.

According to some embodiments, expression of has-miR-486, has-miR-151-3p, has-miR-378, has-miR-122, has-miR-199a-5p; and has-miR-340 are significantly differentially expressed between placenta previa and placenta percreta groups.

According to some embodiments, miRNAs in small-EVs purified from plasma of women with placenta percreta indicated an overall decrease in miRNA expression.

According to some embodiments, the top 14 miRNAs upregulated in placenta percreta play a role in regulation of genes involved in cell migration, cell proliferation and angiogenesis. According to some embodiments, the genes include AKT1, IFGR1, TP53, PIK3C2A, ZEB1, and FOX01.

According to some embodiments, the top 9 down-regulated miRNAs in placenta percreta play a role in regulation of genes involved in cell proliferation, migration and sprouting angiogenesis. According to some embodiments the genes include KRAS, GSK3B, and CCND1.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1: Specificity of EV-CATCHER™ customized antibodies and characterization of small-EVs. FIG. 1A shows Western blots displaying the specificity of the monoclonal placental alkaline phosphatase antibody used for EV-CATCHER™ customization versus alkaline phosphatase, tissue-nonspecific isozyme antibody. FIG. 1B is a schematic depicting the utilization of the EV-CATCHER™ assay customized with a high specific placental alkalkine phosphatase (PLAP) antibody for selectively purifying placenta-specific EVs from maternal blood. FIG. 1C shows representative transmission electron microscopy (TEM) images of small-EVs isolated from normal and placenta previa maternal plasma. FIG. 1D shows representative ONi super resolution images of small-EVs isolated from normal and placenta previa maternal plasma.

FIG. 2: Mean gestation age of plasma samples from placenta previa and percreta subjects. Clinical data on PAS plasma samples was collected at Hackensack University Medical Center (HUMC), including mean gestational age. Prenatal diagnoses were confirmed by histopathological determination after delivery of the placenta following elective cesarean section (placenta previa) or after cesarean hysterectomy (placenta percreta).

FIG. 3: Small-RNA sequencing of placental small-EVs purified from maternal plasma using PLAP+EV-CATCHER™. FIG. 3A is a heatmap representation displaying miRNA expression differences between total RNA extracted from PLAP+ small-EVs from previa (lanes 1-2, 5-12 and 15-20) and percreta (lanes 3-4, 13-14 and 21-32). FIG. 3B shows individual box plot analyses of the 40 differentially expressed miRNAs identified from total RNA extracted from PLAP+ small-EVs purified from maternal plasma with the EV-CATCHER™ assay. FIG. 3C shows a box plot representation of the integrative miRNA signature (40 miRNAs) between total RNA extracted from PLAP+ small-EVs purified EV-CATCHER™ from subjects with placenta previa (n=16) and placenta percreta (n=16).

FIG. 4 shows identification of the top 6 miRNAs differentially expressed in placenta percreta. It provides individual box plot analyses of the 6 top differentially expressed miRNAs identified from total RNA extracted from PLAP+ small-EVs purified from maternal plasma with the EV-CATCHER™ assay (hsa-miR-486, hsa-miR-151-3p, hsa-miR-199a-5p, hsa-miR-378, hsa-miR-122, hsa-miR-340).

FIG. 5 illustrates miRNA enrichment and gene ontology analyses of top upregulated and downregulated miRNAs in placenta percreta. miRNet network analyses displaying the predicted microRNA-gene interactions for the FIG. 5A top 14 upregulated and FIG. 5B the top 9 downregulated miRNAs identified by NGS. Each analysis utilized miTRarBase v8.0 as selected gene target database and filtering was performed using a degree filter of 2.0 for all network nodes and the minimum network selection. REVIGO gene ontology tree maps displaying the predicted biological processes show that the FIG. 5C top 14 upregulated and FIG. 5D top 9 downregulated miRNAs differentially expressed miRNAs are involved. Comprehensive lists of GO terms were obtained from miRBase for all mature differentially expressed miRNAs, overlapping GO terms were consolidated and imputed into REVIGO.

FIG. 6. The utilization of the EV-CATCHER™ assay customized with a high specific placental alkaline phosphatase (PLAP) antibody for the isolation of fetal small-EVs from maternal plasma for the diagnosis of placenta accreta spectrum (PAS) disorders.

FIG. 7 is Table 1, which contains demographic information for the patients from whom 16 plasma samples from placenta percreta and 16 plasma samples from uncompleted placenta previa controls were obtained for the clinical study described in Example 1.

### DETAILED DESCRIPTION OF THE INVENTION

### Definitions

As used herein and in the appended claims, the singular forms “a”, “an”, and “the” include plural reference unless the context clearly dictates otherwise. Thus, for example, reference to a “peptide” is a reference to one or more peptides and equivalents thereof known to those skilled in the art, and so forth.

The term “antibody” as used herein refers to a polypeptide or group of polypeptides comprised of at least one binding domain that is formed from the folding of polypeptide chains having three-dimensional binding spaces with internal surface shapes and charge distributions complementary to the features of an antigenic determinant of an antigen.

The basic structural unit of a whole antibody molecule consists of four polypeptide chains, two identical light (L) chains (each containing about 220 amino acids) and two identical heavy (H) chains (each usually containing about 440 amino acids). The two heavy chains and two light chains are held together by a combination of noncovalent and covalent (disulfide) bonds. The molecule is composed of two identical halves, each with an identical antigen-binding site composed of the N-terminal region of a light chain and the N-terminal region of a heavy chain. Both light and heavy chains usually cooperate to form the antigen binding surface. Human antibodies show two kinds of light chains, K and 2; individual molecules of immunoglobulin generally are only one or the other.

An antibody may be an oligoclonal antibody, a polyclonal antibody, a monoclonal antibody, a chimeric antibody, a CDR-grafted antibody, a multi-specific antibody, a bi-specific antibody, a catalytic antibody, a chimeric antibody, a humanized antibody, a fully human antibody, an anti-idiotypic antibody, and an antibody that can be labeled in soluble or bound form, as well as fragments, variants or derivatives thereof, either alone or in combination with other amino acid sequences provided by known techniques. An antibody may be from any species.

Monoclonal antibodies (mAbs) can be generated by fusing mouse spleen cells from an immunized donor with a mouse myeloma cell line to yield established mouse hybridoma clones that grow in selective media. A hybridoma cell is an immortalized hybrid cell resulting from the in vitro fusion of an antibody-secreting B cell with a myeloma cell. In vitro immunization, which refers to primary activation of antigen-specific B cells in culture, is another well-established means of producing mouse monoclonal antibodies.

Diverse libraries of immunoglobulin heavy (VH) and light (Vκ and Vλ) chain variable genes from peripheral blood lymphocytes also can be amplified by polymerase chain reaction (PCR) amplification. Genes encoding single polypeptide chains in which the heavy and light chain variable domains are linked by a polypeptide spacer (single chain Fv or scFv) can be made by randomly combining heavy and light chain V-genes using PCR. A combinatorial library then can be cloned for display on the surface of filamentous bacteriophage by fusion to a minor coat protein at the tip of the phage.

The technique of guided selection is based on human immunoglobulin V gene shuffling with rodent immunoglobulin V genes. The method entails (i) shuffling a repertoire of human λ light chains with the heavy chain variable region (VH) domain of a mouse monoclonal antibody reactive with an antigen of interest; (ii) selecting half-human Fabs on that antigen (iii) using the selected λ light chain genes as “docking domains” for a library of human heavy chains in a second shuffle to isolate clone Fab fragments having human light chain genes; (v) transfecting mouse myeloma cells by electroporation with mammalian cell expression vectors containing the genes; and (vi) expressing the V genes of the Fab reactive with the antigen as a complete IgG1, λ antibody molecule in the mouse myeloma.

The term antibody also includes binding fragments of the antibodies of the invention; exemplary fragments include Fv, Fab, Fab′, single stranded antibody (svFC), dimeric variable region (Diabody) and di-sulphide stabilized variable region (dsFv). Structural and functional domains can be identified by comparison of the nucleotide and/or amino acid sequence data to public or proprietary sequence databases. For example, computerized comparison methods can be used to identify sequence motifs or predicted protein conformation domains that occur in other proteins of known structure and/or function. Methods to identify protein sequences that fold into a known three-dimensional structure are known. See, for example, Bowie et al. Science 253:164 (1991), which is incorporated by reference in its entirety.

As used herein, the terms “antigen” refers to any substance that elicits an immune response.

The term “antigen-binding site” as used herein refers to the site at the tip of each arm of an antibody that makes physical contact with an antigen and binds it noncovalently. The antigen specificity of the antigen-binding site is determined by its shape and the amino acids present.

The term “antigenic determinant” or “epitope” as used herein refers to that portion of an antigenic molecule that is contacted by the antigen-binding site of a given antibody or antigen receptor.

The terms “residue” or “amino acid residue” or “amino acid” are used interchangeably to refer to an amino acid that is incorporated into a protein, a polypeptide, or a peptide, including, but not limited to, a naturally occurring amino acid and known analogs of natural amino acids that can function in a similar manner as naturally occurring amino acids.

A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. Means for making this adjustment are well-known to those of skill in the art. See, e.g., Pearson (1994) Methods Mol. Biol. 24:307-331, herein incorporated by reference. Examples of groups of amino acids that have side chains with similar chemical properties include (1) aliphatic side chains: glycine, alanine, valine, leucine and isoleucine; (2) aliphatic-hydroxyl side chains: serine and threonine; (3) amide-containing side chains: asparagine and glutamine; (4) aromatic side chains: phenylalanine, tyrosine, and tryptophan; (5) basic side chains: lysine, arginine, and histidine; (6) acidic side chains: aspartate and glutamate, and (7) sulfur-containing side chains are cysteine and methionine.

The term “binding” and its other grammatical forms as used herein means a lasting attraction between chemical substances. Binding specificity involves both binding to a specific partner and not binding to other molecules. Functionally important binding may occur at a range of affinities from low to high, and design elements may suppress undesired cross-interactions. Post-translational modifications also can alter the chemistry and structure of interactions. “Promiscuous binding” may involve degrees of structural plasticity, which may result in different subsets of residues being important for binding to different partners. “Relative binding specificity” is a characteristic whereby in a biochemical system a molecule interacts with its targets or partners differentially, thereby impacting them distinctively depending on the identity of individual targets or partners.

As used herein, the term “binding agent” refer to a substance that can bind to a chemical or other substance, e.g., an antigen.

As used herein, the term “biological particle” refers to a minute portion, piece, fragment or amount (particle) derived from an organism. Biological particles include, without limitation, exosomes, extracellular vesicles, viral particles, bacterial particles, or other secreted particles comprising surface membranes.

The term “biomarker” (or “biosignature”) as used herein refers to peptides, proteins, nucleic acids, antibodies, genes, metabolites, or any other substances used as indicators of a biologic state. It is a characteristic that is measured objectively and evaluated as a cellular or molecular indicator of normal biologic processes, pathogenic processes, or pharmacologic responses to a therapeutic intervention. The term “indicator” as used herein refers to any substance, number or ratio derived from a series of observed facts that may reveal relative changes as a function of time; or a signal, sign, mark, note or symptom that is visible or evidence of the existence or presence thereof. Once a proposed biomarker has been validated, it may be used to diagnose disease risk, presence of disease in an individual, or to tailor treatments for the disease in an individual (e.g., choices of drug treatment or administration regimes).

The term “blastocyst” as used herein refers to the modified blastula stage of mammalian embryos, consisting of the inner cell mass and a thin trophoblast layer enclosing the blastocele.

The term “cargo” as used herein refers to a load or that which is conveyed. With respect to exosomes and/or extracellular vesicles, the term cargo refers to a substance encapsulated in the exosome and/or extracellular vesicle. The compound or substance can be, e.g., a nucleic acid (e.g., nucleotides, DNA, RNA), a polypeptide, a lipid, a protein, or a metabolite, or any other substance that can be encapsulated in an exosome and/or extracellular vesicle. With respect to exosomes and/or extracellular vesicles, the term “cargo profile” as used herein refers to the measurement of the abundance of cargo components (e.g., a nucleic acid (e.g., nucleotides, DNA, RNA), a polypeptide, a lipid, a protein, or a metabolite) that characterize the population of exosomes and/or extracellular vesicles.

The term “cDNA library” as used herein refers to a collection of cloned DNA sequences that are complementary to the mRNA that was extracted from an organism or tissue.

The term “click chemistry” as used herein refers to chemical synthetic methods for making compounds using reagents that can be joined together using efficient reagent conditions and that can be performed in benign solvents or solvents that can be removed or extracted using facile methods, such as evaporation, extraction, or distillation. Several types of reactions that fulfill these criteria have been identified, including nucleophilic ring opening reactions of epoxides and aziridines, non-aldol type carbonyl reactions, such as formation of hydrazones and heterocycles, additions to carbon-carbon multiple bonds, such as oxidative formation of epoxides and Michael additions, and cycloaddition reactions. A representative example of click chemistry is a reaction depicted in Formula I below that couples an azide and an alkyne to form a triazole. The copper-catalyzed azide-alkyne cycloaddition (CuAAC) features an enormous rate acceleration of 107 to 108 compared to the uncatalyzed 1,3-dipolar cycloaddition. It succeeds over a broad temperature range, is insensitive to aqueous conditions and pH range over 4 to 12 and tolerates a broad range of functional groups. Pure products can be isolated by simple filtration or extraction without the need for chromatography or recrystallization.

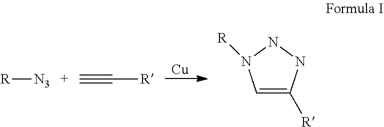

A representative example of copper-free click chemistry is a reaction that couples a dibenzocyclo-octyl (DBCO)-tagged DNA molecule to an azide-functionalized surface by cycloaddition without copper as shown in Formula II:

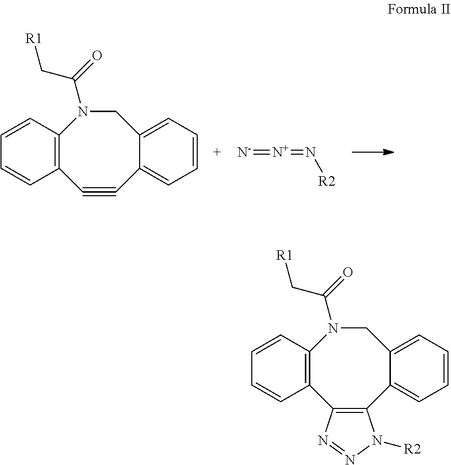

[Eeftens, J M, et al. BMC Biophys. (2015) 8:9].

The term “clickable functional group” as used herein refers to a functional group that can be used in click chemistry to form a product. According to some embodiments, the clickable functional group is an azide or an alkyne.

The term “contact” and its various grammatical forms as used herein refers to a state or condition of touching or of immediate or local proximity.

The term “encapsulated” as used herein refers to being enclosed in a capsule (meaning a membranous envelope enclosing a part).

The term “exosomes and/or extracellular vesicles” as used herein refers to extracellular bilayered membrane-bound vesicles of endosomal origin in a size range of ˜40 to 160 nm in diameter (˜100 nm on average) generated by all cells that are actively secreted.

Biogenesis. Exosomes and/or extracellular vesicles are generated in a process that involves double invagination of the plasma membrane and the formation of intracellular multivesicular bodies (MVBs) containing intraluminal vesicles (ILVs). ILVs are ultimately secreted as exosomes and/or extracellular vesicles with a size range of ˜40 to 160 nm in diameter through MVB fusion to the plasma membrane and exocytosis. The first invagination of the plasma membrane forms a cup-shaped structure that includes cell-surface proteins and soluble proteins associated with the extracellular milieu. This leads to the de novo formation of an early-sorting endosome (ESE) and in some cases may directly merge with a preexisting ESE. The trans-Golgi network and endoplasmic reticulum can also contribute to the formation and the content of the ESE (Kalluri, R., LeBleu, VS. Science (2020) 367 (6478): caau6977, citing Kalluri, R. J. Clin. Invest. (2016) 126:1208-15; van Neil, G. et al. Nat. Rev. Mol. Cell Biol. (2018) 19:213-28; McAndrews, KM, Kalluri, R. Mol. Cancer (2019) 18:52; Mathieu, M. et al. Nat. Cell Biol. (2019) 21:9-17; Willms, E. et al. Front. Immunol. (2018) 9:738; Hessvik, NP, Llorente, A. Cell Mol. Life Sci. (2018) 75:193-208). ESEs can mature into late-sorting endosomes (LSEs) and eventually generate MVBs, which are also called multivesicular endosomes. MVBs form by inward invagination of the endosomal limiting membrane (that is, double invagination of the plasma membrane). This process results in MVBs containing several ILVs (future exosomes and/or extracellular vesicles). The MVB can either fuse with lysosomes or autophagosomes to be degraded or fuse with the plasma membrane to release the contained ILVs as exosomes and/or extracellular vesicles [Id., citing Kahler, C., Kalluri, R. J. Mol. Med. (2013) 91:431037].

Heterogeneity: The heterogeneity of extracellular vesicles is thought to be reflective of their size, content, functional impact on recipient cells, and cellular origin. During their secretion they acquire surface proteins from their cell of origin. They naturally transport mRNA, miRNA, and proteins between cells.

Biomarkers. There is general agreement that their membranes are specifically enriched in tetraspanins CD9, CD37, CD63, CD81, and CD82.

Role. Extracellular vesicles are mediators of near and long-distance intercellular communication in health and disease and affect various aspects of cell biology.

The term “expressing” or “expression” as used herein means the transcription and translation of a nucleic acid molecule by a cell.

The term “extracellular vesicles (EVs)” as used herein refers to nanosized, membrane-bound vesicles released from cells that can transport cargo-including DNA, RNA, and proteins-between cells as a form of intercellular communication. Different EV types, including microvesicles (MVs), exosomes, oncosomes, and apoptotic bodies, have been characterized on the basis of their biogenesis or release pathways. Microvesicles bud directly from the plasma membrane, are 100 nanometers (nm) to 1 micrometer (μm) in size, and contain cytoplasmic cargo (Zaborowski, M P et al. BioScience (2015) 65 (8): 783-97, citing Heijnen, H F et al. Blood (1999) 94:3791-99). Another EV subtype, exosomes, is formed by the fusion between multivesicular bodies and the plasma membrane, by which multivesicular bodies release smaller vesicles (exosomes) whose diameters range from 40 to 160 nm (Id., citing El Andaloussi, S. et al. Nature Reviews Drug Discovery (2013) 12:347-57; Cocucci, E. and Meldolesi J. Trends in Cell Biology (2015) 25:364-72). Dying cells release vesicular apoptotic bodies (50 nm-2 μm) that can be more abundant than exosomes or MVs under specific conditions and can vary in content between biofluids (Id., citing Thery, C. et al. J. Immunology (2001) 1666:7309-18; El Andaloussi, S. et al. Nature Reviews Drug Discovery (2013) 12:347-57). Membrane protrusions can also give rise to large EVs, termed oncosomes (1-10 μm), which are produced primarily by malignant cells in contrast to their nontransformed counterparts (Id., citing Di Vizio, D. et al. Am. J. Pathol. (2012) 181:1573-84; Morello, M. et al. Cell Cycle (2013) 12:3526-36).

The term “Fab fragment” as used herein refers to an antibody fragment composed of a single antigen-binding arm of an antibody without the Fc region, produced by cleavage of IgG by the enzyme papain. It contains the complete light chain plus the amino-terminal variable region and first constant region of the heavy chain, held together by an interchain disulfide bond.

The term “F(ab′)₂ fragment” as used herein refers to an antibody fragment composed of two linked antigen-binding arms (Fab fragments) without the Fc regions, produced by cleavage of IgG with pepsin.

The term “fragment” or “peptide fragment” as used herein refers to a small part derived, cut off, or broken from a larger peptide, polypeptide or protein, which retains the desired biological activity of the larger peptide, polypeptide or protein. Antibody binding fragments (e.g., Fab, Fab′, F(ab′)₂, Fv, and single-chain (sc) antibodies) can be produced by recombinant DNA techniques, or by enzymatic or chemical cleavage of intact antibodies.

The term “gene” as used herein refers to a locatable segment of a genomic sequence corresponding to a unit of inheritance, which is associated with regulatory regions, transcribed regions that code for a protein or RNA product, and other functional sequence regions.

The terms “gene expression” and “expression” are used interchangeably herein to refer to the process by which inheritable information from a gene, such as a DNA sequence, is made into a functional gene product, such as protein or RNA.

Gene Ontology. The goal of the Gene Ontology Consortium is to produce a structured, precisely defined, common, controlled vocabulary for describing the roles of genes and gene products in any organism that can be applied to all eukaryotes even as knowledge of gene and protein roles in cells is accumulating and changing. An ontology comprises a set of well-defined terms with well-defined relationships. The structure itself reflects the current representation of biological knowledge as well as serving as a guide for organizing new data. Data can be annotated to varying levels depending on the amount and completeness of available information. [Ashburner, M. et al. Nat. Genet. (2000) 25 (1): 25-29].

Biological process, molecular function and cellular component are all attributes of genes, gene products or gene-product groups. Each of these may be assigned independently. The relationships between a gene product (or gene-product group) to biological process, molecular function and cellular component are one-to-many, reflecting the biological reality that a particular protein may function in several processes, contain domains that carry out diverse molecular functions, and participate in multiple alternative interactions with other proteins, organelles or locations in the cell. [Ashburner, M. et al. Nat. Genet. (2000) 25 (1): 25-29].

The term “biological process” refers to a biological objective to which the gene or gene product contributes. A process is accomplished via one or more ordered assemblies of molecular function. Processes often involve a chemical or physical transformation, in the sense that something goes into a process and something different comes out of it. [Ashburner, M. et al. Nat. Genet. (2000) 25 (1): 25-29].

“Molecular function” is defined as the biochemical activity (including specific binding to ligands or structures0 of a gene product. This definition also applies to the capability that a gene product (or gene product complex) carries as a potential. It describes only what is done without specifying where or when the event actually occurs. [Ashburner, M. et al. Nat. Genet. (2000) 25 (1): 25-29].

“Cellular component” refers to the place in the cell where a gene product is active. [Ashburner, M. et al. Nat. Genet. (2000) 25 (1): 25-29].

The ontologies are developed for a generic eukaryotic cell. GO terms are connected into nodes of a network, thus the connections between its parents and children are known and form what are technically described as directed acyclic graphs. The ontologies are dynamic, in the sense that they exist as a network that is changed as more information accumulates but have sufficient uniqueness and precision so that databases based on the ontologies can automatically be updated as the ontologies mature. [Ashburner, M. et al. Nat. Genet. (2000) 25 (1): 25-29].

The GO concept is intended to make possible, in a flexible and dynamic way, the annotation of homologous gene and protein sequences in multiple organisms using a common vocabulary that results in the ability to query and retrieve genes and proteins based on their shared biology. [Ashburner, M. et al. Nat. Genet. (2000) 25 (1): 25-29].

The term “genetic engineering” as used herein refers to the use of molecular biology methods to manipulate nucleic acid sequences and introduce nucleic acid molecules into host organisms. The term “genetically engineered” as used herein means a cell that has been subjected to recombinant DNA manipulations, such as the introduction of exogenous nucleic acid molecule, resulting in a cell that is in a form not found originally in nature.

The term “gene product” as used herein refers to a macromolecule (either RNA or protein) produced through the processes of transcription and translation from the expression of a gene.

The term “heterogeneous” as used herein refers to being composed of unrelated or unlike elements or parts; varied; miscellaneous; of different kinds; differing or opposite in structure, quality etc; dissimilar.

The term “homogeneous” as used herein refers to being of the same character, structure, quality; etc.; essentially like; of the same nature; composed of similar or identical elements or parts; uniform.

The term “isolated” is used herein to refer to material, such as, but not limited to, a nucleic acid, peptide, polypeptide, or protein, which is: (1) substantially or essentially free from components that normally accompany or interact with it as found in its naturally occurring environment. The terms “substantially free” or “essentially free” are used herein to refer to more than about 95%, 96%, 97%, 98%, 99% or 100% free. The isolated material optionally comprises material not found with the material in its natural environment; or (2) if the material is in its natural environment, the material has been synthetically (non-naturally) altered by deliberate human intervention to a composition and/or placed at a location in the cell (e.g., genome or subcellular organelle) not native to a material found in that environment. The alteration to yield the synthetic material may be performed on the material within, or removed, from its natural state.

The term “join” and its other grammatical forms as used herein means to link, couple, or connect one thing with another. Each of these terms is used interchangeably with the others.

The term “long noncoding RNA” (“lncRNAs”) as used herein refers to a class of transcribed RNA molecules that are longer than 200 nucleotides and yet do not encode proteins. LncRNAs can fold into complex structures and interact with proteins, DNA and other RNAs, modulating the activity, DNA targets or partners of multiprotein complexes. Crosstalk of lncRNAs with miRNAs creates an intricate network that exerts post-transcriptional regulation of gene expression. For example, lncRNAs can harbor miRNA binding sites and act as molecular decoys or sponges that sequester miRNAs away from other transcripts. Competition between lncRNAs and miRNAs for binding to target mRNAs has been reported and leads to de-repression of gene expression (Zampetaki, A. et al. Front. Physiol. (2018) doi.org/10.3389/fphys.2018.01201, citing Yoon, J H et al. Semin. Cell Dev. Bio. (2014) 34:9-14; Ballantyne, M D et al. Clin. Pharmacol. Ther. (2016) 99:494-501). Finally, lncRNAs may contain embedded miRNA sequences and serve as a source of miRNAs (Id., citing Piccoli, M T et al. Cir. Res. (2017) 121:575-83).

The term “messenger RNA” (“mRNA”) as used herein refers to a coding RNA, which functions in protein translation.

The term “microRNA” (or “miRNA”) as used herein refers to a class of small, 18- to 28-nucleotide-long, noncoding RNA molecules. Their major role is in the posttranscriptional regulation of protein expression

The term “non-coding RNA” (“ncRNA”) as used herein refers to a functional RNA molecule that is transcribed from DNA but not translated into proteins. They are classified into housekeeping and regulatory noncoding RNAs. Housekeeping ncRNAs include ribosomal RNA (rRNA, the RNA component of ribosomes), transfer RNA (tRNA, which functions as an adapter for matching amino acids to mRNA), small nuclear RNA (snRNA, which functions in RNA processing such as mRNA splicing), and small nucleolar RNAs (snoRNAs, which functions in guiding chemical modification of other RNAs). Regulatory noncoding RNAs are divided into short ncRNAs (<200 nt) and long ncRNAs (>200 nts). Short noncoding RNAs <200 nt include microRNA (miRNA), small interfering RNAs (siRNAs) and piwi-associated RNAs (piRNAs), and long noncoding RNAs (>200 nt). [Losko, M. et al. Mediators of Inflammation (2016) 1-12. 10.1155/2016/5365209].

The term “nucleic acid” as used herein refers to a deoxyribonucleotide or ribonucleotide polymer in either single- or double-stranded form, and, unless otherwise limited, encompasses known analogues having the essential nature of natural nucleotides in that they hybridize to single-stranded nucleic acids in a manner similar to naturally occurring nucleotides (e.g., peptide nucleic acids).

The term “overexpressed” as used herein refers to increased quantity of a gene or gene product relative to a quantity of the gene or gene product under normal conditions.

The term “particle” as used herein refers to an extremely small constituent, e.g., microparticles (particles in the micrometer size range), nanoparticles (particles in the nanometer size range), etc.

The term “placenta” as used herein refers to an organ of metabolic interchange between a mammalian fetus and mother. It has a portion of embryonic origin, derived from a highly developed area of the outermost embryonic membrane (chorion frondosum) and a maternal portion formed by a modification of the part of the uterine mucosa (decidua basalis) in which the chorionic vesicle is implanted. Within the placenta, the chorionic villi, with their contained capillaries carrying blood of the embryonic circulation, are exposed to maternal blood in the intervillous spaces in which the villi lie; no direct mixing of fetal and maternal blood occurs, but the intervening tissue (the placental membrane) is sufficiently thin to permit the absorption of nutritive materials, oxygen and some harmful substances, like viruses, into the fetal blood and the release of carbon dioxide and nitrogenous waste from it. At term, the human placenta is disk-shaped, about 4 cm in thickness and 18 cm in diameter, and averages about ⅙ to 1/7 the weight of the fetus; its fetal surface is smooth, being formed by the adherent amnion, with the umbilical cord normally attached near its center. The maternal surface of a detached placenta is rough because of the torn decidual tissue adhering to the chorion and shows lobular elevations called cotyledons or lobes.

The term “placenta accreta” as used herein refers to attachment of the placenta into the muscular wall of the uterus (myometrium) without intervening decidua.

The term “placenta increta” as used herein refers to invasion of the trophoblast into the muscular wall of the uterus (myometrium).

The term “placenta percreta” as used herein refers to invasion of the trophoblast through the myometrium, serosa (meaning the outermost of the extraembryonic membranes that encloses the embryo and all its other membranes; frequently called the trophoderm), and into surrounding structures and tissues.

The term “placenta previa” as used herein refers to a condition in which the placenta is implanted in the lower segment of the uterus, extending to the margin of the internal os of the cervix or completely obstructing the os.

The term “placental alkaline phosphatase” or “PLAP” refers to an enzyme normally produced by primordial germ cells and syncytiotrophoblasts; the detection of its expression has been useful in the diagnosis of germ cell tumors. PLAP immunoreactivity in normal human adult and fetal muscle tissue has been observed. This immunoreactivity seems to relate to the degree of myogenic differentiation in soft tissue tumors and is more frequently expressed in cells with skeletal muscle differentiation and least in those with myofibroblastic features. (Goldsmith, J D, et al. Am J. Surgical Pathol (2002) 26 (12): 1627-33).

The term “plasma” as used herein refers to the fluid (noncellular) portion of circulating blood, and the fluid portion of lymph.

The terms “polypeptide” and “protein” are used herein in their broadest sense to refer to a sequence of subunit amino acids, amino acid analogs, or peptidomimetics. The subunits are linked by peptide bonds, except where noted. These terms also apply to amino acid polymers in which one or more amino acid residue is an artificial chemical analogue of a corresponding naturally occurring amino acid, as well as to naturally occurring amino acid polymers. The terms also are inclusive of modifications including, but not limited to, glycosylation, lipid attachment, sulfation, gamma-carboxylation of glutamic acid residues, hydroxylation and ADP-ribosylation. It will be appreciated, as is well known, that polypeptides may not be entirely linear. For instance, polypeptides may be branched as a result of ubiquitination, or they may be circular, with or without branching, generally as a result of posttranslational events, whether by natural processing or by events brought about by human manipulation, which do not occur naturally. Circular, branched and branched circular polypeptides may be synthesized by entirely synthetic methods.

The term “purification” and its various grammatical forms as used herein refers to a process of isolating or freeing from foreign, extraneous, or objectionable elements. The composition is nonetheless substantially pure in that it has been substantially separated from the substances with which it may be associated in living systems or during synthesis. As used herein, the term “substantially pure” refers purity of at least 75%, at least 80%, at least 85%, at least 90%, at least 95% or at least 99% pure as determined by an analytical protocol. Such protocols may include, for example, without limitation, flow cytometry, electrophoresis, small-RNA sequencing, quantitative PCR, nanoparticle tracking, electron microscopy, mass spectrometry, Western blotting, ELISA, and various metabolic assays.

A recombinant or “engineered” nucleic acid molecule is a nucleic acid molecule that has been altered through human manipulation. As non-limiting examples, a recombinant nucleic acid molecule: 1) includes conjoined nucleotide sequences that are not conjoined in nature, 2) has been engineered using molecular cloning techniques such that it lacks one or more nucleotides with respect to the naturally occurring nucleic acid molecule sequence, or 3) has been manipulated using molecular cloning techniques such that it has one or more sequence changes or rearrangements with respect to the naturally occurring nucleic acid sequence.

A recombinant” cell or vector is one that has been modified by the introduction of a heterologous nucleic acid or a cell that is derived from a cell so modified. Recombinant cells express genes that are not found in identical form within the native (non-recombinant) form of the cell or express native genes that are otherwise abnormally expressed, under-expressed or not expressed at all as a result of deliberate human intervention. The term “recombinant” as used herein does not encompass the alteration of the cell or vector by naturally occurring events (e.g., spontaneous mutation, natural transformation transduction/transposition) such as those occurring without deliberate human intervention.

The term “recombinant expression cassette” refers to a nucleic acid construct, generated recombinantly or synthetically, with a series of specified nucleic acid elements which permit transcription of a particular nucleic acid in a host cell. The recombinant expression cassette can be incorporated into a plasmid, chromosome, mitochondrial DNA, virus, or nucleic acid fragment. Typically, the recombinant expression cassette portion of an expression vector includes, among other sequences, a nucleic acid to be transcribed, a promoter, and a transcription termination signal such as a poly-A signal.

The term “recombinant host” refers to any prokaryotic or eukaryotic cell that contains either a cloning vector or an expression vector. This term also includes those prokaryotic or eukaryotic cells that have been genetically engineered to contain the cloned genes, or gene of interest, in the chromosome or genome of the host cell.

When applied to organisms, the term recombinant, engineered, or genetically engineered refers to organisms that have been manipulated by the introduction of a heterologous or recombinant nucleic acid sequence into the organism, and includes gene knockouts, targeted mutations and gene replacement, promoter replacement, deletion, or insertion, as well as introduction of transgenes into the organism. The heterologous or recombinant nucleic acid molecule can be integrated into the recombinant/genetically engineered organism's genome or in other instances are not integrated into the recombinant/genetically engineered organism's genome.

The term “recombinant protein” as used herein refers to a protein produced by genetic engineering.

The term “serum” as used herein refers to the fluid portion of the blood obtained after removal of the fibrin clot and blood cells.

The term “subject” or “individual” or “patient” are used interchangeably to refer to a member of an animal species of mammalian origin, including but not limited to, mouse, rat, cat, goat, sheep, horse, hamster, ferret, pig, dog, guinea pig, rabbit and a primate, such as, for example, a monkey, ape, or human.

The term “trophoblast” as used herein refers to the mesectodermal cell layer covering the blastocyst that erodes the uterine mucosa and through which the embryo receives nourishment from the mother; the cells do not enter into the formation of the embryo itself but contribute to the formation of the placenta. The trophoblast develops processes that later receive a core of vascular mesoderm and are then known as the chorionic villi; the trophoblast soon becomes two-layered, differentiating into the syncytiotrophoblast, an outer layer consisting of a multinucleated protoplasmic mass (syncytium), and the cytotrophoblast, the inner layer next to the mesoderm in which the cells retain their membranes.

The term “underexpressed” as used herein refers to decreased quantity of a gene or gene product relative to the quantity of a gene or gene product under normal conditions.

A “variant” of a peptide or protein is a peptide or protein sequence that varies at one or more amino acid positions with respect to the reference peptide or protein. A variant can be a naturally occurring variant or can be the result of spontaneous, induced, or genetically engineered mutation(s) to the nucleic acid molecule encoding the variant peptide or protein. A variant peptide can also be a chemically synthesized variant.

### EMBODIMENTS

According to one aspect, the present disclosure provides a non-invasive method for early diagnosis of a placental pathology comprising an abnormal formation or arrangement of a placenta in a uterus of a mammalian female subject during pregnancy, the method comprising selectively purifying from plasma of maternal blood a population of small extracellular vesicles (small-EVs) expressing a placenta-specific surface biomarker; wherein the extracellular vesicles comprise micro-RNA cargo; determining a cargo profile for the small EVs by extracting RNA from the purified population of small EVs; identifying and quantifying expression of small non-coding RNAs comprising one or more micro RNAs (miRNAs) encapsulated by the purified population of exosomes; and comparing the miRNA profile of the placenta specific EVs to the miRNA profile of a healthy control of the same approximate gestational age; wherein the early diagnosis can lead to an improved maternal outcome.

According to some embodiments of the method, the placental pathology includes placenta previa and placenta accrete spectrum (PAS). According to some embodiments, the placenta accrete spectrum (PAS) comprises placenta accreta, placenta increta, and placenta percreta.

According to some embodiments, the method comprises an initial ultrafiltration step, an ultracentrifugation step or both to provide a pooled heterogeneous population of biological particles. As defined herein, biological particles include, without limitation, exosomes, extracellular vesicles, viral particles, bacterial particles, or other secreted particles comprising surface membranes.

According to some embodiments, the purified population of small-EVs is at least 50% pure, at least 55% pure, at least 60% pure, at least 65% pure, at least 70% pure, at least 75% pure, at least 80% pure, at least 85% pure, at least 90% pure, at least 91%, 92%, 93%, 94%, 95%, 96%, 97%, 98% 99% or 100% pure.

According to some embodiments, the purified population of small-EVs is homogeneous.

According to some embodiments, the selective purifying of placenta-specific EVs from maternal blood is by antibody capture of the placental EVs in the maternal plasma.

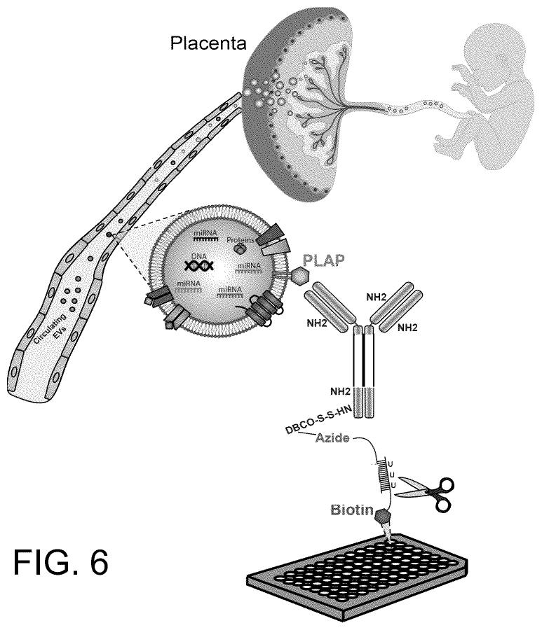

According to some embodiments, the selective purifying of placenta-specific EVs from materinal blood by antibody capture of the placental EVs in the material plasma is via a customized EV-CATCHER™ assay (FIG. 6).

According to some embodiments, the antibody is a monoclonal antibody raised against a recombinant human PLAP and the placenta-specific biomarker comprises a transmembrane placental alkaline phosphatase (PLAP) protein; the monoclonal antibody raised against the recombinant human PLAP is activated with a dibenzocyclo-octyl (DBCO)-ester; the DBCO-modified antibody is coupled to a DNA linker by click chemistry, the antibody-DNA linker conjugates are bound to streptavidin coated well plates pretreated with RNAse A; the purified population of placenta-specific small EVs are released from the streptavidin-coated well plates enzymatically by uracil glycosylase; and the purified population of placenta-specific small-EVs is eluted from the monoclonal PLAP antibody complex by contacting the complex with free PLAP.

According to some embodiments, the method differentiates between small EVs of human women with the placental pathology placenta previa and human women with the placental pathology placenta percreta

According to some embodiments, the method identifies 40 differentially expressed miRNAs, including miR-21 and, miR-191 and miR-223 with increased expression and miR-451 and miR-486 with decreased expression.

According to some embodiments, expression of has-miR-486, has-miR-151-3p, has-miR-378, has-miR-122, has-miR-199a-5p; and has-miR-340 are significantly differentially expressed between placenta previa and placenta percreta groups.

According to some embodiments, miRNAs in small-EVs purified from plasma of women with placenta percreta indicated an overall decrease in miRNA expression.

According to some embodiments, the top 14 miRNAs upregulated in placenta percreta play a role in regulation of genes involved in cell migration, cell proliferation and angiogenesis. According to some embodiments, the genes include AKT1, IFGR1, TP53, PIK3C2A, ZEB1, and FOX01.

The AKT1 gene provides instructions for making AKT1 kinase.

The IFGR1 gene encodes the ligand-binding chain (alpha) of the gamma interferon receptor.

The TP53 gene encodes a tumor suppressor protein containing transcriptional activation, DNA binding, and oligomerization domains.

The protein encoded by the PIK3C2A gene belongs to the phosphoinositide 3-kinase (PI3K) family. PI3-kinases play roles in signaling pathways involved in cell proliferation, oncogenic transformation, cell survival, cell migration, and intracellular protein trafficking.

The ZEB1 gene encodes a zinc finger transcription factor.

The Fox01 gene belongs to the forkhead family of transcription factors which are characterized by a distinct forkhead domain. Fox01 is a cell-specific core transcription factor for endometrial remodeling and homeostasis during the menstrual cycle and early pregnancy.

According to some embodiments, the top 9 down-regulated miRNAs in placenta percreta play a role in regulation of genes involved in cell proliferation, migration and sprouting angiogenesis. According to some embodiments, the genes include KRAS, GSK3β, and CCND1.

The KRAS gene, a Kirsten aras oncogene homolog from the mammalian ras gene family, encodes a protein that is a member of the small GTPase superfamily. Ras proteins bind GDP/GTP and possess intrinsic GTPase activity. GTPase Kras plays an important role in the regulation of cell proliferation.

The GSK3β gene encodes a serine-threonine kinase belonging to the glycogen synthase kinase subfamily. It is a negative regulator of glucose homeostasis and is involved in energy metabolism, inflammation, ER-stress, mitochondrial dysfunction, and apoptotic pathways.

The CCND1 gene encodes cyclin D1, Cyclins function as regulators of cyclin-dependent kinases, which are involved in many crucial processes, such as cell cycle and transcription, as well as communication, metabolism, and apoptosis.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges which may independently be included in the smaller ranges also is encompassed within the invention subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

As used herein and in the appended claims, the singular forms “a”, “and”, and “the” include plural referents unless the context clearly dictates otherwise. All technical and scientific terms used herein have the same meaning.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be confirmed independently.

### EXAMPLES

The following examples are put forth so as to provide those of ordinary skill in the art with a complete disclosure and description of how to make and use the present invention and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

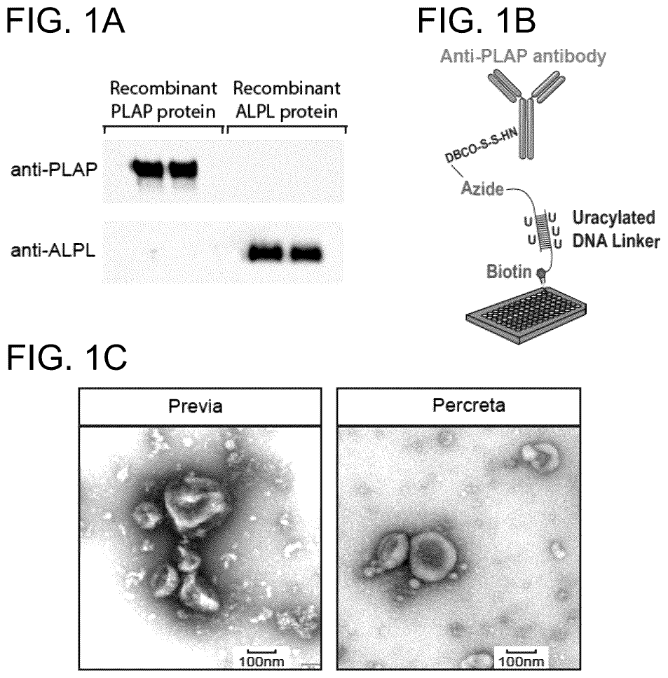

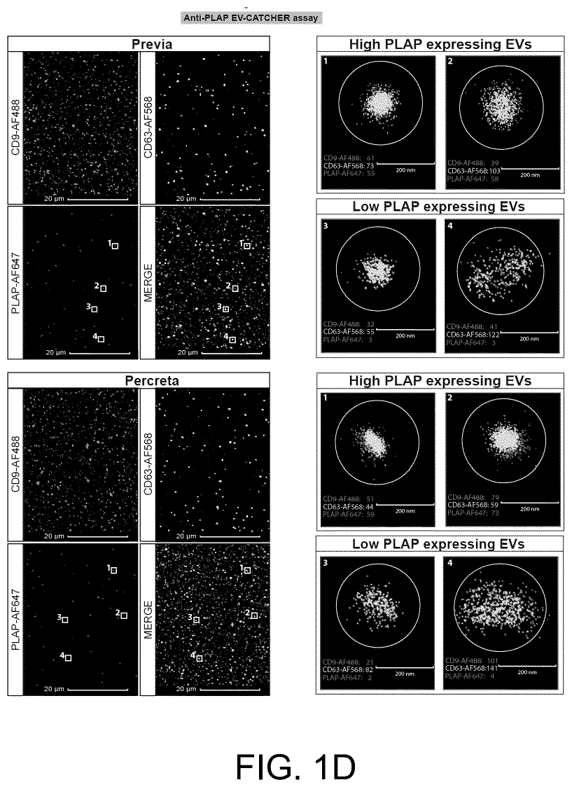

Since we established the state-of-the-art ultra-sensitive antibody-based EV purification assay, termed EV-CATCHER™, which we demonstrated provides ultra-pure EVs when compared to several commercial magnetic bead-based purification assays and thereby increasing biomarker signal to noise ratio, we sought to evaluate the microRNA (miRNA) profiles of placental-specific EVs circulating in maternal blood between two abnormal placental conditions; placenta previa and placenta percreta. To establish a threshold of detection, we evaluated the detection of differentially expressed miRNAs, contained within isolated placental EVs, between these two conditions. For these analyses we compare purified placental EV miRNAs from mothers with placenta previa (i.e., controls; low lying placenta but not abnormally invasive) and mothers with placenta percreta (cases; most severe form of placenta accreta spectrum where the placenta fully invades the uterus with potential for invasion of the bladder). We selected placenta previa controls, to match the same gestational age, which were also histopathologically confirmed after delivery. Our cases were histopathologically confirmed as Grade 3 (percreta) based on histological criteria defined in the recent FIGO Clinical Classification System [9]. To ensure the specific purification of circulating placental EVs, we performed our EV-CATCHER™ assay antibody purifications customized with a monoclonal antibody raised against human-PLAP (FIG. 1B) and utilized a small-RNA cDNA library preparation protocol specifically tailored to investigate the differential expression of miRNAs between our cases and controls [64].

### Materials and Methods

### Clinical Specimen Collection

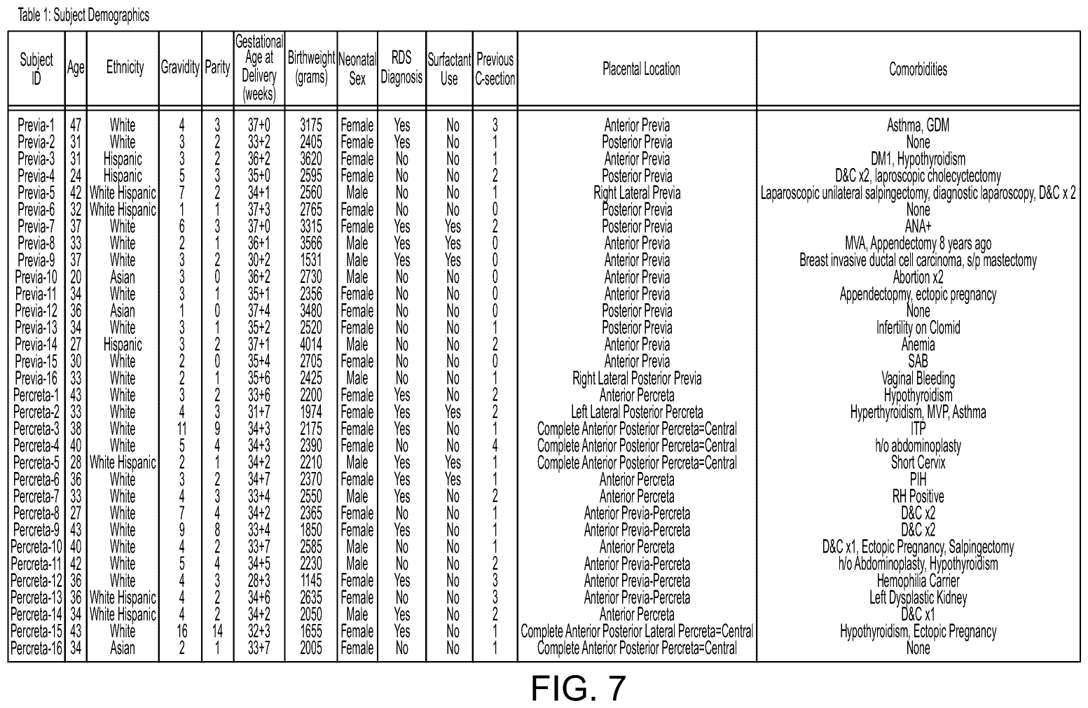

Plasma specimens from patients with placenta percreta (cases) and placenta previa without accreta (previa; controls) obtained immediately prior to delivery, were collected at Hackensack University Medical Center (HUMC) under IRB approval that was acquired prior to the start of the study. All subjects were consented for use of blood samples, and maternal samples were matched where cases and controls had the same approximate gestational age (33-35 weeks). 16 plasma samples from each placenta percreta and uncomplicated placenta previa controls were processed at the Obstetrics and Gynecology laboratory at Jurist research building at HUMC within 1 h of sample collection. FIG. 7 is Table 1, which contains demographic information for the patients from whom 16 plasma samples from placenta percreta and 16 plasma samples from uncompleted placenta previa controls were obtained for the clinical study described in Example 1.

Briefly maternal blood specimens were collected in K-EDTA blood collection tubes, mixed and stored on ice prior to being centrifuged at 2500×g for 10 min. The plasma layer was removed and stored as 500 μl aliquots at −80° C. Prior to final stratification placenta previa and percreta cases were identified by antenatal ultrasound and MRI assessment. In the first trimester of pregnancy ultrasound findings include implantation of the gestational sac in the lower uterine segment, uterine scar area, multiple irregular vascular spaces noted within the placental bed. During the second and third pregnancy trimesters, the placental invasion reflects as turbulent blood flow in placental lacunar spaces, thinning of retroplacental hypoechogenic line or its absence, uneven thinning of the myometrium, the intervention of placental tissue into the posterior wall of the bladder with uneven thinning of uterine and bladder gap and bright blood flow. Distinctive MRI signs of the invasive placenta are intensive heterogeneous placental signals, dark intraplacental bands on T2-weighted images, abnormal placental vascularity, local interruptions in the myometrial wall, and directly visible placental tissue invasion into the nearby pelvic tissues, especially into the bladder, and the prenatal diagnoses were later confirmed by histopathological determination subsequent to delivery of the placenta following elective cesarean section (placenta previa) or cesarean hysterectomy (placenta accreta, increta/percreta).

### Western Blot Analysis

Western blot analyses were conducted to confirm the specificity of the PLAP antibody used to purify PLAP+ small-EVs from maternal plasma. Purified recombinant proteins from both placental specific alkaline phosphatase (PLAP, #NBP2-52266) and alkaline phosphatase, tissue-nonspecific isozyme (ALPL, #2909-AP-010) were purchased from Novus biologicals and separated on 4-12% polyacrylamide precast mini-PROTEAN TGX gel (Bio-Rad, #4561086) by sodium dodecyl sulphate polyacrylamide gel electrophoresis (SDS-PAGE). 5 μl of Precision Plus Protein™ Dual Xtra Prestained Protein Standard (Bio-Rad, #1610377) was loaded and used for gel orientation and determination of molecular weights of separated proteins. 10 μg of each purified recombinant protein was loaded, and gels were run at 100 V and 400 mA for 90 min (Power Pac 300, Bio-Rad) in 1×Tris/Glycine/SDS buffer (Bio-Rad, #1610732). After the SDS-PAGE run, proteins were transferred to 0.2 μm polyvinylidene fluoride (PVDF) membranes (Bio-Rad, #1704156) using a semi-dry electro-transfer system (TransBlot Turbo v1.02, Bio-Rad) for 30 min at 25 V. Membranes were visualized using the stain-free blot protocol provided on a Chemi-Doc™ MP (Bio-Rad) system to evaluate protein transfer and membranes were blocked using EveryBlot blocking buffer (Bio-Rad, #12010020) for 30 min. Membranes were incubated at 4° C. O/N with TBS-T (1×TBS, pH 6.8, 0.1% Tween20) diluted anti-mouse primary antibodies (1:1000) targeted against PLAP (Novus Biological, #NBP2-47993) and ALPL (Novus Biologicals, #NBP2-22193). Membranes were washed with TBS-T (3× 5 min) before incubation in anti-mouse IgG horseradish peroxidase conjugated secondary antibodies (1:10000) for 1 h, with gentle agitation at RT. Membranes were washed with TBS-T (3×5 min) before proteins were detected using SuperSignal™ West Femto Maximum Sensitivity Substrate (Pierce, #34095) and protein bands were visualized using ImageLab 4.0 software on a Chemi-Doc MP (Bio-Rad) imaging system.

### EV-CATCHER™ Isolation of PLAP+ Small-Extracellular Vesicles

The isolation of placenta specific small-EVs was performed using the EV-CATCHER™ isolation protocol described by Mitchell et al., 2021 using the placenta specific PLAP (placental alkaline phosphatase) as the capture antibody and as illustrated in FIG. 1B and FIG. 6. Briefly, equimolar amounts (1:1 ratios) of oligonucleotides (Integrated DNA Technologies) 5′-Azide (AAAAACGAUUCGAGAACGUGACUGCCAUGCCAGCUCGUACUAUCGAA) and 3′-Biotin (GAUAGUACGAGCUGGCAUGGCAGUCACGUUCUCGAAUCGUUUU) were annealed (90° C. for 2 min, 90-42° C. for 40 min, 42° C. for 120 min) in 1×RNA annealing buffer (60 mM KCl, 6 mM HEPES (pH 7.5), 0.2 mM MgCl2), prior to separation on a 15% non-denaturing polyacrylamide (PAGE) gel (450 volts for 90 min). The double stranded (ds) DNA linker was visualized on a blue light box with SYBR® Gold™ dye (ThermoFisher, #S11494), excised, centrifugally crushed using a gel breaker tube (IST Engineering, #3388-100) and resuspended in 400 mM NaCl and shaken overnight (O/N) on a thermomixer set to 4° C. and 1,100 RPM. The solution was filtered, and the dsDNA linker was purified using the QIAEX® II gel extraction kit (Qiagen #20021) according to manufacturer instructions. The anti-PLAP antibody (1 mg/ml) used for small-EV pulls (Novus, #NBP2-47993), was activated using 5 μl of freshly prepared 4 mM DBCO-NHS ester (Lumiprobe, #94720) and incubated for 30 min at room temperature (RT) in the dark, reactions were stopped by adding 2.5 μl of 1 M Tris-Cl (pH 8.0) at RT for 5 min in the dark. DBCO-activated anti-PLAP was then desalted onto pre-equilibrated Zeba desalting columns (ThermoFisher, #89882) by incubation for 1 min and centrifugation at 1,500×g for 2 min. Purified dsDNA linker and DBCO activated anti-PLAP antibody were quantified on a Nanodrop 2000 instrument prior to the preparation of antibody-dsDNA (Ab-dsDNA) stock solutions where 100 μg of activated antibody was conjugated to 50 μg of purified DNA linker, and incubated O/N at 4° C. on a rotator. The next day, Ab-dsDNA conjugates were bound to streptavidin coated 96-well plates (Pierce #15120) by incubating 1 μg of anti-PLAP antibody (linker bound) in 100 μl 1×PBS per well (2 wells were prepared per sample), plates were then placed on a plate shaker at 300 RPM at 4° C., for 8 h to allow for binding to the plate. Solutions were carefully removed, and wells were washed three times with cold 1×PBS solution, prior to addition of RNase-A (12.5 μg/ml) treated samples (100 μl). Plates were sealed using microAMP optical adhesive film (Applied Biosystems, #4311971) and placed on a shaker at 300 RPM at 4° C., O/N. Samples were carefully removed, wells were washed 3 times with cold 1×PBS and 100 μl of freshly prepared uracil glycosylase (UNG) enzyme (ThermoFisher, #EN0362) in 1×PBS (1×UNG buffer (200 mM Tris-Cl (pH 8.0), 10 mM EDTA and 100 mM NaCl), with 1 unit of enzyme) was added to each well. Plates were incubated at 37° C. for 2 h on a shaker at 300 RPM for UNG digestion of the dsDNA linker, and PLAP+ small-EVs were recovered in this solution for downstream miRNA analyses.

### Transmission Electron Microscopy

Transmission electron microscopy (TEM) of small-EVs was performed at the analytical imaging facility at the Albert Einstein College of Medicine. Briefly, purified small-EVs were fixed using 2% Glutaraldehyde in phosphate buffer (Electron Microscopy Services #6536-05) and stored at 4° C. 300 mesh formvar-coated grids were inverted onto 20 μl of fixed small-EV suspensions for approximately 2 min and wicked dry. Grids were then inverted onto 40 μl of 2% aqueous uranyl acetate for approximately 1 min, and wicked dry. Samples were imaged on a JEOL JEM-1400+ transmission electron microscope (JEOL Ltd.; Tokyo, Japan) operating at an accelerating voltage of 80 kV. High resolution TIFF images were acquired and saved using an AMT 16 MP digital camera system (Advanced Microscopy Techniques Corp.; Woburn, MA).

### ONi Super Resolution Nanoimaging

Purified PLAP+ small-EVs were processed for imaging on the ONi super resolution Nanoimager using the ONi EV Profiler kit v2.0 according to manufacturer's instructions. Briefly, the surface of the assay capture chip was prepared by applying 5 μl of S3 buffer to each lane and incubated at room temperature for 10 min. 30 μl of W1 was then applied to each lane to remove excess S3, after which 10 μl of S4 buffer was slowly pipetted to each lane ensuring that no bubbles were introduced into lanes. After a 10-minute incubation period at room temperature lanes were again washed by applying 30 μl of W1 buffer to each lane. EV capture was then performed by immediately applying 10 μL of EV-CATCHER™ purified PLAP+ small-EVs and allowing binding to occur for 15 min. Lanes were then washed using 30 μl of W1 buffer and captured EVs were fixed by adding 20 μl of F1 to each lane and incubating the chip at room temperature for 10-minutes. The staining of captured small-EVs was performed by firstly preparing a three-antibody working solution comprised of CD63-Fluor® 568, CD9-Alexa Fluor® 488 and PLAP-Alexa Fluor® 647 antibodies combined together in W1 buffer so that each antibody is at a dilution of 1:20. The final staining solution is prepared by combining 1 μl of the prepared working solution with 9 μl of NI buffer for each lane, gently pipetting to mix the solution and applying 10 μl to each lane of the EV profiler chip and allowed to incubate for 50 minutes at room temperature in the dark. Immediately following antibody incubation lanes were washed with 30 μl of W1 buffer followed by a 20-minute incubation with 20 μl of F1 buffer for 10 minutes. A final wash step was performed and BCubed™ dSTORM imaging buffer added to each well immediately before EV profiler chips were imaged. Image acquisition on the ONi super resolution Nanoimager was performed in the NimOS Light program with a 640 dichroic split using the following parameters: 640 nm laser set to 20-30% laser power, the 560 nm laser at 35% laser power and the 473/488 nm laser set to 70% laser power. The number of runs (frames) for all laser lines was set to 1000 and all image analyses were performed using CODI software.

### RNA Extractions

PLAP+ small-EVs isolated from maternal plasma were subjected to total RNA extraction using the miRNeasy Serum/Plasma kit (Qiagen, Cat #217184) according to manufacturer's instructions with some modifications to improve total RNA yield. Briefly, QIAzol was added to 100 μl of PLAP+ small-EVs, vortexed and incubated at RT for 5 min, after which chloroform was added to each sample. Samples were vortexed again and incubated at RT for 5 min. Samples were centrifuged at 12,000×g, at 4° C. for 15 min and the upper aqueous phase of each sample was carefully removed and transferred into new siliconized tubes, to which 100% ethanol was added. Samples were incubated on ice for 40 min prior to column purification. The clear upper phase was then passed twice through supplied RNeasy minElute columns, washed with RPE, and ice cold 80% ethanol. Columns were spun to remove residual ethanol and total RNA was eluted with 50 μl of RNase-free water. Samples were then speed-vacuumed to 10 μl prior to small-RNA sequencing.

Small-RNA cDNA Library Preparations

Small-RNA sequencing from small-EVs was performed using the cDNA library preparation protocol described by Loudig et al. (2017), with modifications for low input RNA from purified small-EVs [65,66]. For PLAP+ small-EVs purified from previa and percreta plasma, the small-RNA cDNA library preparation was performed using total RNA recovered from small-EVs purified from 200 μl of plasma (2 wells were prepared for each serum sample (100 μl plasma per well) and isolated small-EVs were pooled for RNA extraction). Samples were divided into two libraries, each containing 8 placenta percreta and 8 previa samples (a total of 16 samples per library). 16 ligations were set up individually by combining 9.5 μl of total RNA, 8.5 μl of the master-mix and 1 μl of 50 μM adenylated barcoded 3′ adapter (Integrated DNA Technologies, custom order). A master-mix was then prepared using 0.0052 nM calibrator cocktail (40 μl 10×RNA ligase-2 buffer, 120 μl 50% DMSO, and 10 μl calibrator cocktail). Reactions were heated at 90° C. for 1 min, incubated on ice for 2 min, and 1 μl ( 1/10 diluted) truncated K227Q T4 RNA Ligase 2 (New England Biolabs #M0351L) was added to each reaction, which were then incubated O/N on ice in a cold room. The next day, ligations were heat inactivated at 90° C. for 1 min, and individually precipitated by addition of 1.2 μl of Glycoblue mix (1 μl Glycoblue™ Co-precipitant (15 mg/ml; ThermoFisher, #AM9516) in 26 μl 5 M NaCl (ThermoFisher, #AM9579)) and 63 μl of 100% ethanol was added to each tube. Reactions were combined, precipitated on ice for 1 h and centrifuged for 1 h at 14,000 RPM, at 4° C. The pellet was dried and resuspended in 20 μl nuclease-free water and 20 μl denaturing PAA gel loading solution and separated on a 15% Urea-PAGE gel. Size marker RNA oligonucleotides (IDT) were used to guide gel excision. The gel piece was crushed using a gel-breaker tube (IST Engineering #3388-100) and incubated in 400 mM NaCl O/N at 4° C., at 1,100 RPM on a thermomixer. The next day the solution was filtered and precipitated in 100% ethanol on ice for 1 h. RNA pellet was obtained by centrifugation at 14,000 RPM for 1 h at 4° C. The 5′ adapter was added to the resuspended pellet and T4 RNA Ligase 1 (New England Biolabs #M0204L) was added for 1 h at 37° C. The ligated product was separated on a 12% Urea-PAGE gel in the presence of 5′ ligated size markers, as guide for size selection. The gel was spun in a gel breaker tube, after which the crushed gel was resuspended in 300 mM NaCl solution with 1 ul 100 M 3′ PCR primer, and incubated O/N on a thermomixer at 1,100 RPM at 4° C. Subsequently, the solution was filtered, precipitated with 100% ethanol, incubated on ice for 1 h and pelleted by centrifugation at 14,000 RPM for 1 h at 4° C. The RNA pellet was resuspended in nuclease free water for reverse transcription (3 μl 5× first strand buffer, 1.5 μl of 0.1 M DTT, and 4.2 μl dNTP Mix (2 mM each; ThermoFisher, #R0241)) with 0.75 μl SuperScript® III Reverse Transcriptase (ThermoFisher, #18080-093) and incubated at 50° C. for 30 min. Reverse transcription was deactivated at 95° C. for 1 min, followed by addition of 95 μl nuclease-free water. A pilot PCR reaction was set up (10 μl 10×PCR buffer, 10 μl dNTP Mix (2 mM each), 10 μl cDNA, 67 μl nuclease-free water, 0.5 μl 3′ PCR primer, 0.5 μl 5′ PCR primer, and 2 μl Titanium® Taq DNA Polymerase (Clontech Laboratories #639208)). 12 μl aliquots were withdrawn at cycles 10, 12, 14, 16, 18, 20 and 22 for analysis on a 2.5% agarose gel, and identification of the optimal PCR amplification cycle. Six PCR reactions were then set up, run for the optimal number of amplification cycles, a portion (10 μl) was evaluated on a 2.5% agarose gel. The remaining solution was combined, precipitated, digested with Pmel for removal of size markers, and separated on a 2.5% gel. The 100 nt PCR library product was excised, purified with QIAquick Gel Extraction Kit (Qiagen #28704) and quantified using the Qubit® dsDNA HS Assay Kit (ThermoFisher, #Q32854). cDNA libraries were then sequenced (single-read 50 cycles) on a HiSeq 2500 Sequencing System (Illumina #SY-401-2501), after which FASTQ files containing raw sequencing data were processed for adapter trimming and small-RNA alignment to the hg-19 genome. Read counts were normalized to total counts and subjected to statistical analyses (see below).

In Silico miRNA Enrichment and Gene Ontology Analyses

MiRNA enrichment analysis (miRNet) and REVIGO gene ontology analyses were performed separately for the top 25 upregulated and the top 15 downregulated miRNAs from the list of top 40 differentially expressed miRNAs obtained from miRNA sequencing. Based on the list of the top 40 differentially expressed miRNAs between percreta cases and previa controls, miRNAs were further stratified by disregarding any miRNAs where the baseMean (readcount) fell below 100. The resulting list of 23 differentially expressed miRNAs were then used for all subsequent enrichment analyses and gene ontology pathway predictions. Based on the fold change statistics generated miRNAs were classified as either upregulated or downregulated and miRBase ID were obtained from the miRBase website. Additionally, for all mature miRNAs GO terms from miRBase were also obtained. Assessment of the top 14 upregulated and top 9 downregulated miRNAs were assessed separately in miRNet (www.mirnet.ca/upload/MirUploadView.xhtml), where each of the upregulated and downregulated miRNA lists were imputed using their miRBase IDs and selecting miTRarBase v8.0 as target. The interaction networks for both the upregulated and downregulated miRNAs were filtered using a degree filter of 2.0 for all network nodes and the minimum network selection was selected. Two separate comprehensive lists (one for upregulated and one for downregulated miRNAs) of GO Terms were obtained from miRBase for all mature differentially expressed miRNAs, overlapping GO terms were consolidated and each list was separately imputed into REVIGO (http://revigo.irb.hr/) to visualize the predicted gene ontology pathways.

### Data Analysis

Raw FASTQ data files obtained on an Illumina HiSeq2500 sequencer were processed using the RNAworld server from the Tuschl Laboratory at the Rockefeller University, including adapter trimming and read alignments and annotation. MiRNA counts were exported to spreadsheets for data analysis. Statistical analyses of miRNA counts were performed using dedicated Bioconductor packages in the R platform, as detailed below. Heat maps were generated from transformed counts using the ‘NMF’ package (aheatmap function). Differential expression was assessed using ‘DESeq2’ and ‘edgeR’. Differential expression models included a batch variable (library) to reduce batch biases. To maximize the discrimination ability of miRNA we computed a score for each sample (miRNA score′, [67]), assembled by summing the standardized levels (z-values) of all significantly upregulated miRNA, and the negative of the z-values of all significantly downregulated miRNA.

### Results

### Validation of PLAP Customization of EV-CATCHER™

In order to determine the specificity of the EV-CATCHER™ customization we performed for the isolation of placenta-specific EVs from maternal blood (FIG. 1B), we tested the monoclonal PLAP antibody we selected against both recombinant placental alkaline phosphatase (PLAP) and a recombinant alkaline phosphatase, tissue-nonspecific isozyme (ALPL) proteins using Western blot analyses. We demonstrate that the anti-PLAP antibody reacted strongly with the recombinant PLAP protein with no cross-reactivity to the recombinant ALPL protein (FIG. 1A). Then as proof-of-concept characterization of the small-EVs we purify using the monoclonal anti-PLAP antibody customized EV-CATCHER™ assay, we isolated small-EVs from maternal plasma from a woman with a normal healthy pregnancy and one diagnosed with placenta previa, using transmission electron microscopy (TEM) imaging and ONi super resolution nanoimaging. Both TEM (FIG. 1C) and ONi super resolution imaging (FIG. 1D) demonstrated that the morphology and size of PLAP+ small-EVs isolated from maternal plasma are consistent with those we previously demonstrated for EV-CATCHER™ isolated small-EVs and what is currently described in the literature for extracellular vesicles isolated from plasma [68].

miRNA Analysis of PLAP+ Small-EVs Purified from Maternal Plasma of Previa and Percreta Patients Using the EV-CATCHER™ Assay.

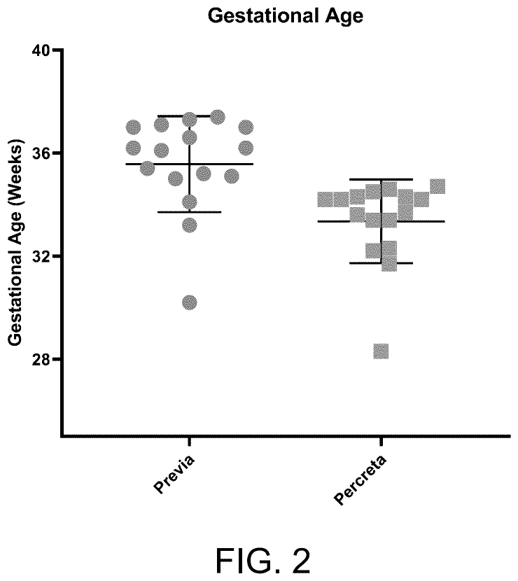

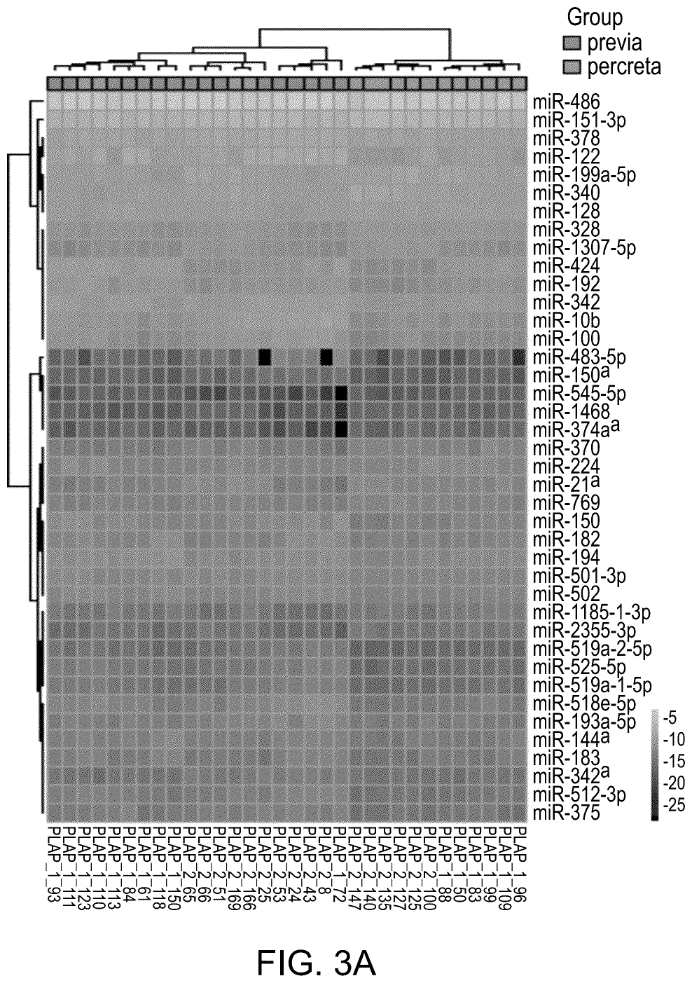

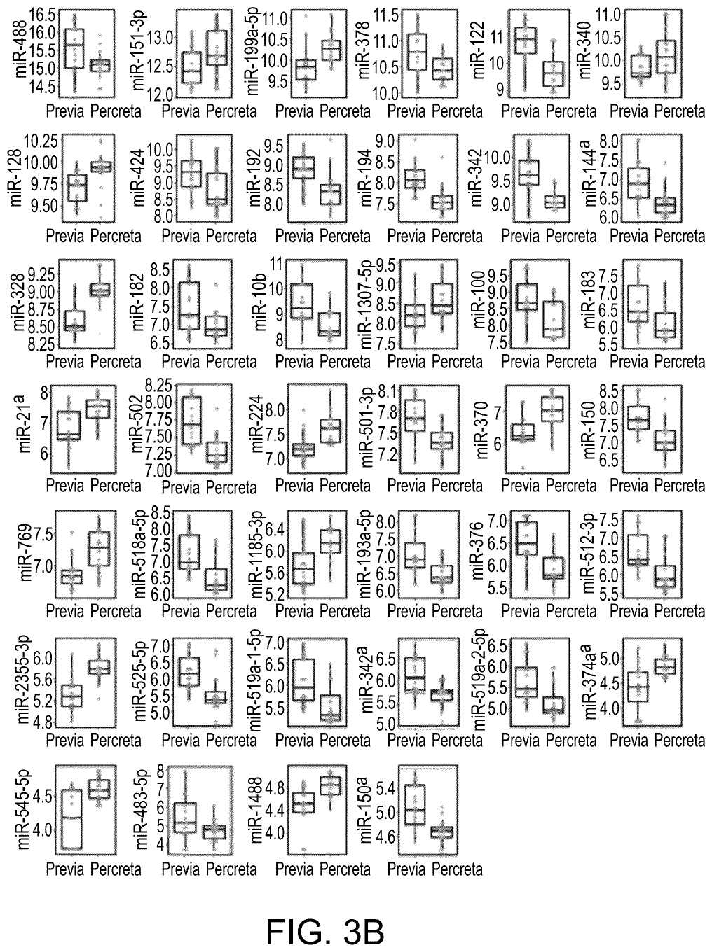

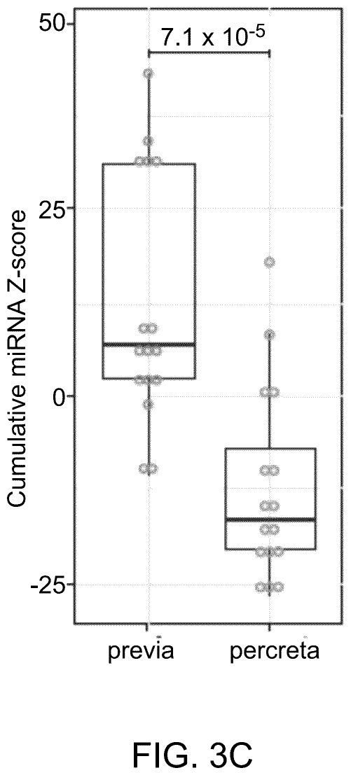

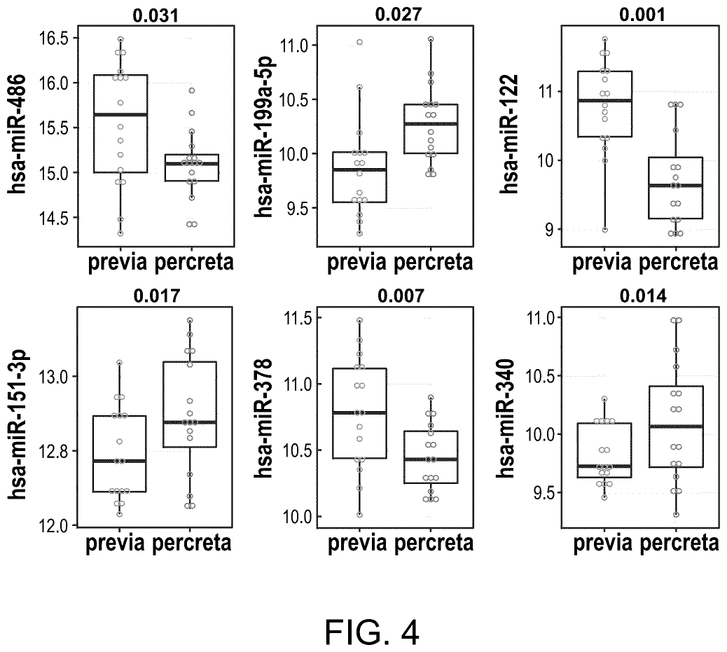

We sought to determine if the placenta-tissue specific customization of our ultra-sensitive purification assay may yield detection of differential miRNA expression changes in small-EVs from placenta previa and placenta percreta maternal plasma samples. Therefore, small-RNA cDNA libraries were prepared using RNA extracted from PLAP+EV-CATCHER™ small-EVs isolated from the plasma of women diagnosed with placenta previa (n=16; controls) and women diagnosed with placenta percreta (cases; n=16), with a mean gestational age of 35±4 weeks for previa and 33±4 weeks for percreta (FIG. 2). We prepared 2 cDNA libraries including the 32 samples, with each library containing an even number of cases and controls, to minimize bias and batch effect. Statistical analyses of our NGS data demonstrated that we were able to differentiate almost perfectly between small-EVs of women with placenta previa and women with placenta percreta using miRNA expression levels and without supervised clustering (FIG. 3A), which indicated the presence of highly differentially expressed miRNAs between the two conditions. Upon further analyses, we identified a total of 40 significantly (pval<0.05) differentially expressed miRNAs between placenta previa and percreta sample groups (FIG. 3B). We established an integrative miRNA signature using the 40 differentially expressed miRNAs extracted from PLAP+ small-EVs, which provided an even greater discrimination between the two groups, as the p-value was estimated at 7.1 e-5. Interestingly, the global expression trend for miRNAs in small-EVs purified from plasma of women with placenta percreta indicated an overall decrease in expression (FIG. 3C). This decrease in overall miRNA expression is biologically compatible with a decrease in mRNA target inhibition and a potential increase of cellular proliferation and invasion. Analysis of the differentially expressed miRNAs, identified 6 significant differentially expressed miRNAs (p<0.05) between previa and percreta sample groups (FIG. 4; hsa-miR-486 (p-val=0.031), hsa-miR-151-3p (p-val=0.017), hsa-miR-378 (p-val=0.007), hsa-miR-122 (p-val=0.001), hsa-miR-199a-5p (p-val=0.027), and hsa-miR-340 (p-val=0.014), which have been described to be associated with oncogenic processes.

miRNA Pathway Enrichment Analysis and Prediction of Biological Pathway involvement Using Gene Ontology (GO).

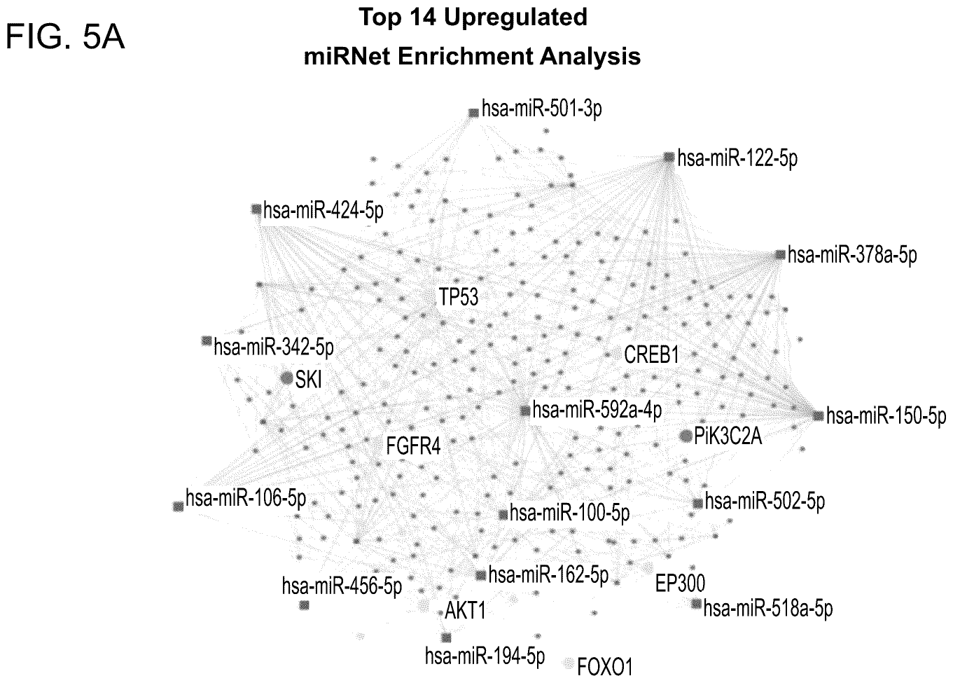

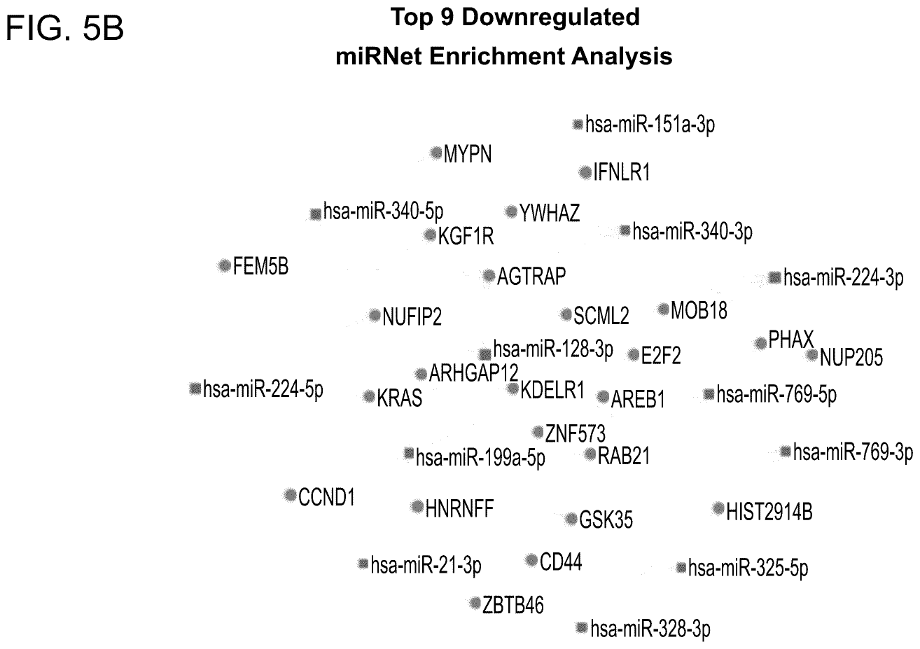

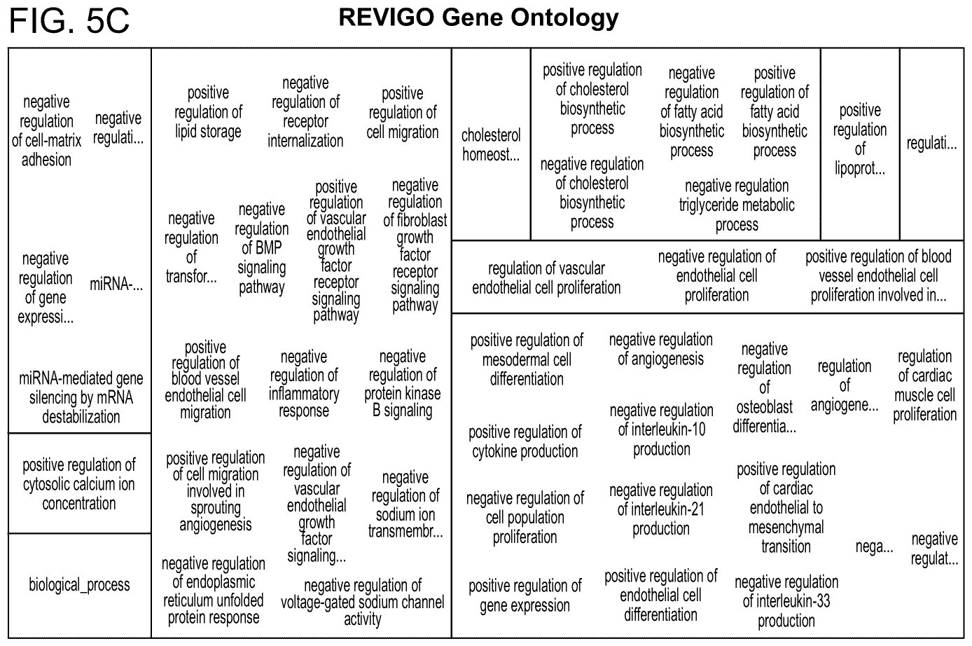

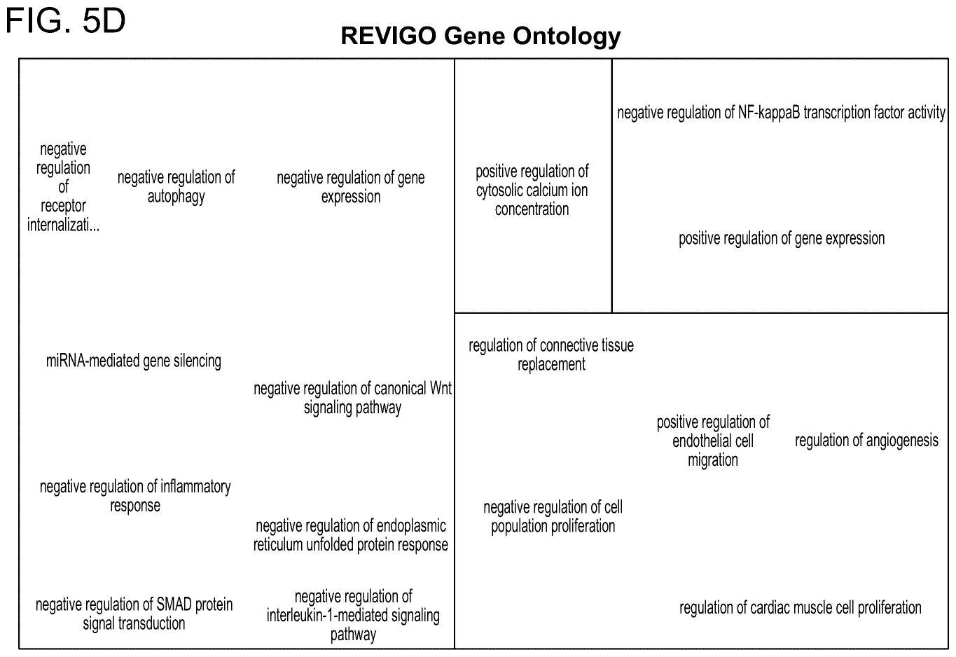

Since miRNAs bind to target genes and regulate their expression through mRNA destabilization and inhibition of gene translation [69], it is possible that for a given miRNA list the biological processes they post-transcriptionally regulate can be predicted using in silico enrichment analysis tools. Our initial miRNA pathway enrichment analyses suggest that the top 14 miRNAs we identified to be upregulated in placenta percreta play a role in the regulation of genes including AKT1, IGFR1, TP53, PIK3C2A, ZEB1 and FOXO1 which are known to be involved in cell migration, cell proliferation and angiogenesis (FIG. 5A). Assessment of the top 9 down regulated miRNAs in placenta percreta similarly show a regulation in cell proliferation, migration and sprouting angiogenesis through their predicted interactions with genes such as KRAS, GSK3ß and CCND1 (FIG. 5B).

### DISCUSSION

In this study we isolated placenta-specific (PLAP+) small-EVs from maternal plasma from women diagnosed with placenta or placenta percreta which were assessed for potential small-RNA biomarkers differences that may help with diagnosis of PAS. This study, which adheres to current clinical and scientific guidelines, used maternal plasma specimens from which the histopathologic confirmation of case diagnosis and differentiation between PAS grades was confirmed and that used the FIGO Clinical Classification System to allow for multi-center standardization. Additionally, appropriate controls (placenta previa), sufficient sample sizes and the absence of overlap between control and case values was determined to minimize analytical bias.

Our analysis of circulating miRNAs contained within placenta-specific EVs of women with placenta previa (controls) or with placenta percreta (cases) identified 40 differentially expressed miRNAs, including miR-21, miR-191 and miR-223 with increased expression and miR-451 and miR-486 with decreased expression. The identification of several differentially expressed miRNAs circulating in the blood of pregnant women is in agreement with previous findings where we observed significant miRNA expression differences between cases and controls when assessing circulating small-EVs isolated from plasma [64]. Based on previous studies, the miRNA profiles obtained from the isolation of small-EVs demonstrates enhanced specificity and signal-to-noise ratio in comparison to that obtained from the assessment of whole plasma, which carries both free-floating miRNAs (cell-free miRNAs combined with argonaute) [70-73] as well as those encapsulated within small-EVs [74-77]. Considering that most cells are known to shed both cell-free miRNAs and small-EVs into the circulation contributes, a high level of surrounding miRNA noise signal is anticipated with whole plasma. Therefore, in this study we performed small-RNA sequencing analysis of PLAP+ small-EVs isolated from maternal plasma of women diagnosed with placenta previa (controls) and placenta percreta (cases) using our customizable EV-CATCHER™ assay. We observed that indeed with the isolation of placenta-specific (PLAP+) small-EVs, we identify an 8-fold increase in the number of differentially expressed miRNAs (40 miRNAs) when compared to whole plasma miRNA analyses. Our analyses highlight not only the need for targeted antibody selection but also for more sensitive approaches for biomarker analyses, especially for the analysis of circulating miRNAs.

Several of the miRNAs we identified to be differentially expressed in percreta small-EVs have previously been shown to be associated with adverse pregnancy outcomes, for example miR-144 is known to regulate trophoblast proliferation, migration and invasion and its deregulation plays a role in the pathophysiology of preeclampsia [78-80], whereas, miR-199a plays a critical role in mediating the opposing effects of estrogen and progesterone in uterine contractility during pregnancy, and the downregulation of miR-199a during pregnancy results in spontaneous preterm birth [81-82]. Many other studies have demonstrated that miR-486 is suppressed in different cancer types, including lung, colorectal, and thyroid carcinoma [83-86], and when transfected into cells leads to suppressed cell migration, denoting its role in angiogenesis and invasion [87], and possibly its role in over invasive placentation. Furthermore, miR-122 downregulation in PAS, has been characterized as to be upregulated in fetuses with intrauterine growth restriction (IUGR) [88].

While the expression changes that were detected for these miRNAs was relatively limited, the number of reads for these miRNAs was very high, amongst the most highly expressed species (within the top 8), with significant p-values. The top 8 miRNAs, which displayed the most significant differences in the plasma samples of the PAS cases all appeared to be involved in the regulation of migration/invasion. Several of these, although not all, have shown these effects in trophoblast cells. The mechanisms by which they achieve these biological effects remain clear, but it appears that small-EVs with these miRNAs provide a significant stimulus to invasion. These results suggest a possible mechanistic role for these differentially expressed miRNAs in pathologies involving aberrant invasion, including PAS, and warrants further evaluation. Of particular note in this regard, 25 of the top 50 miRNAs which were enriched in our PAS samples arise from the placenta-specific chromosome 19 miRNA cluster, suggesting that the placenta is indeed the origin of these small-EV harboring miRNAs. Recently miRNAs from the placenta-specific chromosome 19 cluster have been shown to promote invasion [89].

Our miRNA pathway enrichment and REVIGO gene ontology analyses further support the role of these miRNAs in the regulation of genes that are involved in the promotion of cellular invasion and the progression of PAS. Specifically, our miRNet analysis of the top 14 differentially expressed miRNAs identified to be upregulated in PAS show a strong interaction with ZEB1 which has been shown to play a pivotal role in enabling proliferation, invasion, and EMT of trophoblast cells during pregnancy [90]. Moreover, the predicted interactions with both IGFR1 and AKT1 are associated with cell proliferation, survival, metabolism, protein synthesis, and cell growth in many cell types [91]. The disruption of IGF/insulin signaling has been established as a cause of placental disruption that leads to junctional zone (Jz) defects [92], reduced placental size and fetal growth restriction [93].

Collectively, our data suggests that a large proportion of miRNAs are present in our preparations of PLAP+ small-EVs in both placenta previa (controls) and percreta (cases) samples, and that their expression is significantly different and represents a large miRNA cluster regulating cell proliferation and migration. While previous studies have demonstrated that placenta-specific miRNAs are indeed packaged into small-EVs [94], to our knowledge none have isolated EVs directly from maternal blood. We demonstrate that not only can placenta-specific small-EVs be directly isolated from maternal plasma but that our anti-PLAP customized EV-CATCHER assay has the potential to identify biomarkers for non-invasive detection of PAS. However, further analyses will have to be performed to determine how early on in pregnancy these placenta-specific small-EVs can be captured and assessed to differentiate PAS conditions.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

### REFERENCES

1. Afshar Y, Dong J, Zhao P, Li L, Wang S, Zhang R Y, Zhang C, Yin O, Han C S, Einerson B D, Gonzalez T L, Zhang H, Zhou A, Yang Z, Chou S J, Sun N, Cheng J, Zhu H, Wang J, Zhang T X, Lee Y T, Wang J J, Teng P C, Yang P, Qi D, Zhao M, Sim M S, Zhe R, Goldstein J D, Williams J 3rd, Wang X, Zhang Q, Platt L D, Zou C, Pisarska M D, Tseng H R, Zhu Y. Circulating trophoblast cell clusters for early detection of placenta accreta spectrum disorders. Nat Commun. 2021 Aug. 3; 12 (1): 4408. doi: 10.1038/s41467-021-24627-2. PMID: 34344888.
2. Morlando M, Collins S. Placenta Accreta Spectrum Disorders: Challenges, Risks, and Management Strategies. Int J Womens Health. 2020. 10; 12:1033-1045. doi: 10.2147/IJWH.S224191. PMID: 33204176.
3. Pegu B, Thiagaraju C, Nayak D, Subbaiah M. Placenta accreta spectrum-a catastrophic situation in obstetrics. Obstet Gynecol Sci. 2021. 64 (3): 239-247. doi: 10.5468/ogs.20345. Epub 2021 Mar. 24. PMID: 33757280.
4. American College of Obstetricians and Gynecologists; Society for Maternal-Fetal Medicine. Obstetric Care Consensus No. 7: Placenta Accreta Spectrum. Obstet Gynecol. 2018 December; 132 (6): e259-c275. doi: 10.1097/AOG.0000000000002983. PMID: 30461695.
5. Bartels H C, Postle J D, Downey P, Brennan D J. Placenta Accreta Spectrum: A Review of Pathology, Molecular Biology, and Biomarkers. Dis Markers. 2018 Jul. 3; 2018:1507674. doi: 10.1155/2018/1507674. PMID: 30057649; PMCID: PMC6051104.
6. Bailit J. L., Grobman W. A., Rice M. M., et. al.: Morbidly adherent placenta treatments and outcomes. Obstet Gynecol 2015; 125: pp. 683-689.
7. Fitzpatrick K. E., Sellers S., Spark P., Kurinczuk J. J., Brocklehurst P., Knight M.: The management and outcomes of placenta accreta, increta, and percreta in the UK: a population-based descriptive study. BJOG 2014; 121: pp. 62-71.
8. Silver R M. Abnormal Placentation: Placenta Previa, Vasa Previa, and Placenta Accreta. Obstet Gynecol. 2015 September; 126 (3): 654-668. doi: 10.1097/AOG.0000000000001005. PMID: 26244528.
9. Piñas Carrillo A, Chandraharan E. Placenta accreta spectrum: Risk factors, diagnosis and management with special reference to the Triple P procedure. Womens Health (Lond). 2019 January-December; 15:1745506519878081. doi: 10.1177/1745506519878081. PMID: 31578123; PMCID: PMC6777059.
10. Jauniaux E., Ayres-de-Campos D., Langhoff-Roos J., Fox K. A., Collins S., Diagnosis FPA, Management Expert Consensus P: FIGO classification for the clinical diagnosis of placenta accreta spectrum disorders. Int. J. Gynaecol. Obstet. 2019; 146: pp. 20-24.
11. DaSilva-Arnold S. C., Zamudio S., Al-Khan A., Alvarez-Perez J., Mannion C., Koenig C., Luke D., Perez A. M., Petroff M., Alvarez M., Illsley N. P.: Human trophoblast epithelial-mesenchymal transition in abnormally invasive placenta. Biol. Reprod. 2018; 99: pp. 409-421.
12. Jauniaux E, Bunce C, Grønbeck L, Langhoff-Roos J. Prevalence and main outcomes of placenta accreta spectrum: a systematic review and meta-analysis. Am J Obstet Gynecol. 2019 September; 221 (3): 208-218. doi: 10.1016/j.ajog.2019.01.233. Epub 2019 Feb. 1. PMID: 30716286.
13. Di Mascio D, Calì G, D'antonio F. Updates on the management of placenta accreta spectrum. Minerva Ginecol. 2019 April; 71 (2): 113-120. doi: 10.23736/S0026-4784.18.04333-2. Epub 2018 Nov. 27. PMID: 30486635.
14. Kingdom J C, Hobson S R, Murji A, Allen L, Windrim R C, Lockhart E, Collins S L, Soleymani Majd H, Alazzam M, Naaisa F, Shamshirsaz A A, Belfort M A, Fox K A. Minimizing surgical blood loss at cesarean hysterectomy for placenta previa with evidence of placenta increta or placenta percreta: the state of play in 2020. Am J Obstet Gynecol. 2020 September; 223 (3): 322-329. doi: 10.1016/j.ajog.2020.01.044. Epub 2020 Jan. 30. PMID: 32007492.
15. Jauniaux E, Moffett A, Burton G J. Placental Implantation Disorders. Obstet Gynecol Clin North Am. 2020 March; 47 (1): 117-132. doi: 10.1016/j.ogc.2019.10.002. Epub 2019 Dec. 18. PMID: 32008663.
16. Baldwin H J, Nippita T A, Torvaldsen S, Ibicbele I, Ford J B, Patterson J A. Outcomes of Subsequent Births After Placenta Accreta Spectrum. Obstet Gynecol. 2020 October; 136 (4): 745-755. doi: 10.1097/AOG.0000000000004051. PMID: 32925617.
17. Silver R M, Branch D W. Placenta Accreta Spectrum. N Engl J Med. 2018 Apr. 19; 378 (16): 1529-1536. doi: 10.1056/NEJMcp1709324. PMID: 29669225.
18. Al-Khan A, Youssef Y H, Feldman K M, Illsley N P, Remache Y, Alvarez-Perez J, Mannion C, Alvarez M, Zamudio S. Biomarkers of abnormally invasive placenta. Placenta. 2020 February; 91:37-42. doi: 10.1016/j.placenta.2020.01.007. Epub 2020 Jan. 23. PMID: 32174305.
19. Schwickert A, Chantraine F, Ehrlich L, Henrich W, Muallem M Z, Nonnenmacher A, Petit P, Weizsäcker K, Braun T. Maternal Scrum VEGF Predicts Abnormally Invasive Placenta Better than NT-proBNP: a Multicenter Case-Control Study. Reprod Sci. 2021 February; 28 (2): 361-370. doi: 10.1007/s43032-020-00319-y. Epub 2020 Oct. 6. PMID: 33025531; PMCID: PMC7808970.
20. Berkley E M, Abuhamad A. Imaging of Placenta Accreta Spectrum. Clin Obstet Gynecol. 2018 December; 61 (4): 755-765. doi: 10.1097/GRF.0000000000000407. PMID: 30339609.
21. Chen X, Shan R, Song Q, Wei X, Liu W, Wang G. Placenta percreta evaluated by MRI: correlation with maternal morbidity. Arch Gynecol Obstet. 2020 March; 301 (3): 851-857. doi: 10.1007/s00404-019-05420-5. Epub 2020 Jan. 4. PMID: 31903499.
22. Thiravit S, Lapatikarn S, Muangsomboon K, Suvannarerg V, Thiravit P, Korpraphong P. MRI of placenta percreta: differentiation from other entities of placental adhesive disorder. Radiol Med. 2017 January; 122 (1): 61-68. doi: 10.1007/s11547-016-0689-3. Epub 2016 Sep. 20. PMID: 27651244.
23. Al-Khan A, Gupta V, Illsley N P, Mannion C, Koenig C, Bogomol A, et al. Maternal and fetal outcomes in placenta accreta after institution of team-managed care. Reprod Sci. 2014; 21 (6): 761±71. Epub 2013 Dec. 18. https://doi.org/10.1177/1933719113512528 PMID: 24336676.
24. Wu S, Kocherginsky M, Hibbard J U. Abnormal placentation: twenty-year analysis Am J Obstet Gynecol. 2005; 192 (5): 1458±61. https://doi.org/10.1016/j.ajog.2004.12.074 PMID: 15902137.
25. Mehrabadi A, Hutchcon J A, Liu S, Bartholomew S, Kramer M S, Liston R M, et al. Contribution of placenta accreta to the incidence of postpartum hemorrhage and severe postpartum hemorrhage. ObstetGynecol. 2015; 125 (4): 814±21. https://doi.org/10.1097/AOG.0000000000000722 PMID: 25751202.
26. American College of O, Gynecologists. ACOG committee opinion no. 560: Medically indicated late-preterm and early-term deliveries. Obstet Gynecol. 2013; 121 (4): 908±10. https://doi.org/10.1097/01.AOG.0000428648.75548.00 PMID: 23635709.
27. Robinson B K, Grobman W A. Effectiveness of timing strategies for delivery of individuals with placenta previa and accreta. Obstet Gynecol. 2010; 116 (4): 835±42. Epub 2010 Sep. 23.https://doi.org/10.1097/AOG.0b013e3181f3588d PMID: 20859146.
28. Chantraine F, Blacher S, Berndt S, Palacios-Jaraquemada J, Sarioglu N, Nisolle M, Braun T, Munaut C, Foidart J M. Abnormal vascular architecture at the placental-maternal interface in placenta increta. Am J Obstet Gynecol. 2012 September; 207 (3): 188.el-9. doi: 10.1016/j.ajog.2012.06.083. Epub 2012 Jul. 7. PMID: 22939721.
29. Biberoglu E, Kirbas A, Daglar K, Biberoglu K, Timur H, Demirtas C, Karabulut E, Danisman N. Serum angiogenic profile in abnormal placentation. J Matern Fetal Neonatal Med. 2016 October; 29 (19): 3193-7. doi: 10.3109/14767058.2015.1118044. Epub 2016 Feb. 10. PMID: 26863111.
30. Uyanikoğlu H, İncebiyik A, Turp A B, çakmak G, Sak S, Hilali N G. Serum Angiogenic and Anti-angiogenic Markers in Pregnant Women with Placenta Percreta. Balkan Med J. 2018 Jan. 20; 35 (1): 55-60. doi: 10.4274/balkanmedj.2016.1890. Epub 2017 Sep. 13. PMID: 28903888; PMCID: PMC5820448.
31. Laban M, Ibrahim E A, Elsafty M S, Hassanin A S. Placenta accreta is associated with decreased decidual natural killer (dNK) cells population: a comparative pilot study. Eur J Obstet Gynecol Reprod Biol. 2014 October; 181:284-8. doi: 10.1016/j.ejogrb.2014.08.015. Epub 2014 Aug. 15. PMID: 25195203.
32. Adler E, Madankumar R, Rosner M, Reznik S E. Increased placental trophoblast inclusions in placenta accreta. Placenta. 2014 December; 35 (12): 1075-8. doi: 10.1016/j.placenta.2014.09.014. Epub 2014 Oct. 2. PMID: 25305693.
33. DaSilva-Arnold S C, Zamudio S, Al-Khan A, Alvarez-Perez J, Mannion C, Koenig C, Luke D, Perez A M, Petroff M, Alvarez M, Illsley N P. Human trophoblast epithelial-mesenchymal transition in abnormally invasive placenta. Biol Reprod. 2018 Aug. 1; 99 (2): 409-421. doi: 10.1093/biolre/ioy042. PMID: 29438480.
34. McNally L, Zhou Y, Robinson J F, Zhao G, Chen L M, Chen H, Kim M Y, Kapidzic M, Gormley M, Hannibal R, Fisher S J. Up-regulated cytotrophoblast DOCK4 contributes to over-invasion in placenta accreta spectrum. Proc Natl Acad Sci USA. 2020 Jul. 7; 117 (27): 15852-15861. doi: 10.1073/pnas. 1920776117. Epub 2020 Jun. 23. PMID: 32576693; PMCID: PMC7355036.
35. Berezowsky A, Pardo J, Ben-Zion M, Wiznitzer A, Aviram A. Second Trimester Biochemical Markers as Possible Predictors of Pathological Placentation: A Retrospective Case-Control Study. Fetal Diagn Ther. 2019; 46 (3): 187-192. doi: 10.1159/000492829. Epub 2019 Feb. 6. PMID: 30726846.
36. Lee M S, Cantonwine D, Little S E, McElrath T F, Parry S I, Lim K H, Wilkins-Haug L E. Angiogenic markers in pregnancies conceived through in vitro fertilization. Am J Obstet Gynecol. 2015 August; 213 (2): 212.el-8. doi: 10.1016/j.ajog.2015.03.032. Epub 2015 Mar. 19. PMID: 25797229.
37. Büke B, Akkaya H, Demir S, Sağol S, Şimşek D, Başol G, Barutçuoğlu B. Relationship between first trimester aneuploidy screening test serum analytes and placenta accreta. J Matern Fetal Neonatal Med. 2018 January; 31 (1): 59-62. doi: 10.1080/14767058.2016.1275546. Epub 2017 Jan. 17. PMID: 28027672.
38. Samuel A, Bonanno C, Oliphant A, Batey A, Wright J D. Fraction of cell-free fetal DNA in the maternal serum as a predictor of abnormal placental invasion-a pilot study. Prenat Diagn. 2013 November; 33 (11): 1050-3. doi: 10.1002/pd.4195. Epub 2013 Aug. 4. PMID: 23836321.
39. Zhang J, Li J, Saucier J B, Feng Y, Jiang Y, Sinson J, McCombs A K, Schmitt E S, Peacock S, Chen S, Dai H, Ge X, Wang G, Shaw C A, Mei H, Breman A, Xia F, Yang Y, Purgason A, Pourpak A, Chen Z, Wang X, Wang Y, Kulkarni S, Choy K W, Wapner R J, Van den Veyver I B, Beaudet A, Parmar S, Wong L J, Eng C M. Non-invasive prenatal sequencing for multiple Mendelian monogenic disorders using circulating cell-free fetal DNA. Nat Med. 2019 March; 25 (3): 439-447. doi: 10.1038/s41591-018-0334-x. Epub 2019 Jan. 28. Erratum in: Nat Med. 2019 Feb. 20; PMID: 30692697.
40. Lyell D J, Faucett A M, Baer R J, Blumenfeld Y J, Druzin M L, El-Sayed Y Y, Shaw G M, Currier R J, Jelliffe-Pawlowski L L. Maternal serum markers, characteristics and morbidly adherent placenta in women with previa. J Perinatol. 2015 August; 35 (8): 570-4. doi: 10.1038/jp.2015.40. Epub 2015 Apr. 30. PMID: 25927270.
41. Shainker S A, Silver R M, Modest A M, Hacker M R, Hecht J L, Salahuddin S, Dillon S T, Ciampa E J, D'Alton M E, Otu H H, Abuhamad A Z, Einerson B D, Branch D W, Wylic B J, Libermann T A, Karumanchi S A. Placenta accreta spectrum: biomarker discovery using plasma protcomics. Am J Obstet Gynecol. 2020 September; 223 (3): 433.e1-433.e14. doi: 10.1016/j.ajog.2020.03.019. Epub 2020 Mar. 19. PMID: 32199927.
42. Jiang Q, Dai L, Chen N, Li J, Gao Y, Zhao J, Ding L, Xic C, Yi X, Deng H, Wang X. Integrative analysis provides multi-omics evidence for the pathogenesis of placenta percreta. J Cell Mol Med. 2020 December; 24 (23): 13837-13852. doi: 10.1111/jcmm.15973. Epub 2020 Oct. 21. PMID: 33085209; PMCID: PMC7754008.
43. Gagnon A, Wilson R D; SOCIETY OF OBSTETRICIANS AND GYNAECOLOGISTS OF CANADA GENETICS COMMITTEE. Obstetrical complications associated with abnormal maternal serum markers analytes. J Obstet Gynaecol Can. 2008 October; 30 (10): 918-932. English, French. doi: 10.1016/S1701-2163 (16) 32973-5. PMID: 19038077.
44. Uyanikoglu H, Sak M E, Tatli F, Hilali N G, Sak S, Incebiyik A, Barut M U, Erel O, Gonel A. Scrum ischemia modified albumin level and its relationship with the thiol/disulfide balance in placenta percreta patients. J Obstet Gynaecol. 2018 November; 38 (8): 1073-1077. doi: 10.1080/01443615.2018.1450369. Epub 2018 Jun. 8. PMID: 29884071.
45. Ophir E, Tendler R, Odch M, Khouri S, Oettinger M. Creatine kinase as a biochemical marker in diagnosis of placenta increta and percreta. Am J Obstet Gynecol. 1999 April; 180 (4): 1039-40. doi: 10.1016/s0002-9378 (99) 70683-6. PMID: 10203680.
46. Hilali N, Kocarslan S, Vural M, Incebiyik A, Camuzcuoglu A, Camuzcuoglu H. Ki-67 proliferation index in patients with placenta previa percreta in the third trimester. Wien Klin Wochenschr. 2015 February; 127 (3-4): 98-102. doi: 10.1007/s00508-014-0634-1. Epub 2014 Nov. 15. PMID: 25398292.
47. Chen S, Pang D, Li Y, Zhou J, Liu Y, Yang S, Liang K, Yu B. Scrum miRNA biomarker discovery for placenta accreta spectrum. Placenta. 2020 November; 101:215-220. doi: 10.1016/j.placenta.2020.09.068. Epub 2020 Sep. 29. PMID: 33017714.
48. Mouillet J. F., Ouyang Y., Coyne C. B., Sadovsky Y.: MicroRNAs in placental health and disease. Am. J. Obstet. Gynecol. 2015; 213: pp. S163-S172.
49. Lycoudi A., Mavreli D., Mavrou A., Papantoniou N., Kolialexi A.: miRNAs in pregnancy-related complications. Expert Rev. Mol. Diagn. 2015; 15: pp. 999-1010.
50. Miura K., Miura S., Yamasaki K., Higashijima A., Kinoshita A., Yoshiura K., Masuzaki H.: Identification of pregnancy-associated microRNAs in maternal plasma. Clin. Chem. 2010; 56: pp. 1767-1771.
51. Jin J, Menon R. Placental exosomes: A proxy to understand pregnancy complications. Am J Reprod Immunol. 2018 May; 79 (5):e12788. doi: 10.1111/aji. 12788. Epub 2017 Nov. 28. PMID: 29193463
52. Nakahara A, Nair S, Ormazabal V, Elfeky O, Garvey C E, Longo S, Salomon C. Circulating Placental Extracellular Vesicles and Their Potential Roles During Pregnancy. Ochsner J. 2020 Winter; 20 (4): 439-445. doi: 10.31486/toj.20.0049. PMID: 33408584; PMCID: PMC7755551.
53. Schuster J, Cheng S B, Padbury J, Sharma S. Placental extracellular vesicles and pre-eclampsia. Am J Reprod Immunol. 2021 February; 85 (2):e13297. doi: 10.1111/aji.13297. Epub 2020 Jul. 18. PMID: 32619308; PMCID: PMC7779690.
54. Miranda J, Paules C, Nair S, Lai A, Palma C, Scholz-Romero K, Rice G E, Gratacos E, Crispi F, Salomon C. Placental exosomes profile in maternal and fetal circulation in intrauterine growth restriction-Liquid biopsies to monitoring fetal growth. Placenta. 2018 April; 64:34-43. doi: 10.1016/j.placenta.2018.02.006. Epub 2018 Mar. 2. PMID: 29626979.
55. Salomon, C.; Torres, M. J.; Kobayashi, M.; Scholz-Romero, K.; Sobrevia, L.; Dobierzewska, A.; Illanes, S. E.; Mitchell, M. D.; Rice, G. E. A gestational profile of placental exosomes in maternal plasma and their effects on endothelial cell migration. PLOS ONE 2014, 9, c98667.
56. Karin-Kujundzic, V.; Sola, I. M.; Predavec, N.; Potkonjak, A.; Somen, E.; Mioc, P.; Serman, A.; Vranic, S.; Serman, L. Novel Epigenetic Biomarkers in Pregnancy-Related Disorders and Cancers. Cells 2019, 8, 1459. https://doi.org/10.3390/cells8111459
57. Sussman H H, Bowman M, Lewis J L Jr. Placental alkaline phosphatase in maternal serum during normal and abnormal pregnancy. Nature. 1968 Apr. 27; 218 (5139): 359-60. doi: 10.1038/218359a0. PMID: 5649676.
58. Jin, J.; Menon, R. Placental exosomes: A proxy to understand pregnancy complications. Am. J. Reprod. Immunol. 2018, 79, c12788
59. Sarker, S., Scholz-Romero, K., Perez, A. et al. Placenta-derived exosomes continuously increase in maternal circulation over the first trimester of pregnancy. J Transl Med 12, 204 (2014). https://doi.org/10.1186/1479-5876-12-204
60. Czernek L, Düchler M. Exosomes as Messengers Between Mother and Fetus in Pregnancy. Int J Mol Sci. 2020 Jun. 15; 21 (12): 4264. doi: 10.3390/ijms21124264. PMID: 32549407; PMCID: PMC7352303.
61. Williams J L, Gatson N N, Smith K M, Almad A, McTigue D M, Whitacre C C. Serum exosomes in pregnancy-associated immune modulation and neuroprotection during CNS autoimmunity. Clin Immunol. 2013 November; 149 (2): 236-43. doi: 10.1016/j.clim.2013.04.005. Epub 2013 Apr. 18. PMID: 23706172; PMCID: PMC3778091.
62. Sabapatha A, Gercel-Taylor C, Taylor D D. Specific isolation of placenta-derived exosomes from the circulation of pregnant women and their immunoregulatory consequences. Am J Reprod Immunol. 2006 November-December; 56 (5-6): 345-55. doi: 10.1111/j.1600-0897.2006.00435.x. PMID: 17076679.
63. Mitchell M D, Peiris H N, Kobayashi M, Koh Y Q, Duncombe G, Illanes S E, Rice G E, Salomon C. Placental exosomes in normal and complicated pregnancy. Am J Obstet Gynecol. 2015 October; 213 (4 Suppl): S173-81. doi: 10.1016/j.ajog.2015.07.001. PMID: 26428497.
64. Mitchell M I, Ben-Dov I Z, Liu C, Ye K, Chow K, Kramer Y, Gangadharan A, Park S, Fitzgerald S, Ramnauth A, Perlin D S, Donato M, Bhoy E, Manouchehri Doulabi E, Poulos M, Kamali-Moghaddam M, Loudig O. Extracellular Vesicle Capture by AnTibody of CHoice and Enzymatic Release (EV-CATCHER): A customizable purification assay designed for small-RNA biomarker identification and evaluation of circulating small-EVs. J Extracell Vesicles. 2021 June; 10 (8):e12110. doi: 10.1002/jev2.12110. PMID: 34122779.
65. Loudig O, Liu C, Rohan T, Ben-Dov I Z. Retrospective MicroRNA Sequencing: Complementary DNA Library Preparation Protocol Using Formalin-fixed Paraffin embedded RNA Specimens. J Vis Exp. 2018; (135): 57471. doi: 10.3791/57471. PMID: 29781987.
66. Loudig O, Wang T, Ye K, Lin J, Wang Y, Ramnauth A, Liu C, Stark A, Chitale D, Greenlec R, Multerer D, Honda S, Daida Y, Spencer Feigelson H, Glass A, Couch F J, Rohan T, Ben-Dov I Z. Evaluation and Adaptation of a Laboratory-Based cDNA Library Preparation Protocol for Retrospective Sequencing of Archived MicroRNAs from up to 35-Year-Old Clinical FFPE Specimens. Int J Mol Sci. 2017; 18 (3): 627. doi: 10.3390/ijms18030627. PMID: 28335433.
67. Mazch H, Deutch T, Karas A, Bogardus K A, Mizrahi I, Gur-Wahnon D, Ben-Dov I Z. Next-Generation Sequencing Identifies a Highly Accurate miRNA Panel That Distinguishes Well-Differentiated Thyroid Cancer from Benign Thyroid Nodules. Cancer Epidemiol Biomarkers Prev. 2018; 27 (8): 858-863. doi: 10.1158/1055-9965.EPI-18-0055. PMID: 30049841.
68. Holcar M, Ferdin J, Sitar S, Tušck-Žnidarič M, Dolžan V, Plemenitaš A, Žagar E, Lenassi M. Enrichment of plasma extracellular vesicles for reliable quantification of their size and concentration for biomarker discovery. Sci Rep. 2020 Dec. 7; 10 (1): 21346. doi: 10.1038/s41598-020-78422-y. PMID: 33288809; PMCID: PMC7721811.
69. Bartel D P. MicroRNAs: target recognition and regulatory functions. Cell. 2009 Jan. 23; 136 (2): 215-33. doi: 10.1016/j.cell.2009.01.002. PMID: 19167326; PMCID: PMC3794896.
70. Endzeliņš E, Berger A, Melne V, Bajo-Santos C, Sobolevska K, Ābols A, Rodriguez M, Šantarc D, Rudņickiha A, Lietuvietis V, Llorente A, Linē A. Detection of circulating miRNAs: comparative analysis of extracellular vesicle-incorporated miRNAs and cell-free miRNAs in whole plasma of prostate cancer patients. BMC Cancer. 2017 Nov. 9; 17 (1): 730. doi: 10.1186/s12885-017-3737-z. PMID: 29121858; PMCID: PMC5679326.
71. Murillo O D, Thistlethwaite W, Rozowsky J, Subramanian S L, Lucero R, Shah N, Jackson A R, Srinivasan S, Chung A, Laurent C D, Kitchen R R, Galeev T, Warrell J, Diao J A, Welsh J A, Hanspers K, Riutta A, Burgstaller-Muchlbacher S, Shah R V, Yeri A, Jenkins L M, Ahsen M E, Cordon-Cardo C, Dogra N, Gifford S M, Smith J T, Stolovitzky G, Tewari A K, Wunsch B H, Yadav K K, Danielson K M, Filant J, Moeller C, Nejad P, Paul A, Simonson B, Wong D K, Zhang X, Balaj L, Gandhi R, Sood A K, Alexander R P, Wang L, Wu C, Wong D T W, Galas D J, Van Keuren-Jensen K, Patel T, Jones J C, Das S, Cheung K H, Pico A R, Su A I, Raffai R L, Laurent L C, Roth M E, Gerstein M B, Milosavljevic A. exRNA Atlas Analysis Reveals Distinct Extracellular RNA Cargo Types and Their Carriers Present across Human Biofluids. Cell. 2019 April 4; 177 (2): 463-477.e15. doi: 10.1016/j.cell.2019.02.018. PMID: 30951672; PMCID: PMC6616370.
72. Joosse S A, Müller V, Steinbach B, Pantel K, Schwarzenbach H. Circulating cell-free cancer-testis MAGE-A RNA, BORIS RNA, let-7b and miR-202 in the blood of patients with breast cancer and benign breast diseases. Br J Cancer. 2014 Aug. 26; 111 (5): 909-17. doi: 10.1038/bjc.2014.360. Epub 2014 Jul. 1. PMID: 24983365; PMCID: PMC4150270.
73. Morisaki S, Miura K, Higashijima A, Abe S, Miura S, Hasegawa Y, Yoshida A, Kancuchi M, Yoshiura K, Masuzaki H. Effect of labor on plasma concentrations and postpartum clearance of cell-free, pregnancy-associated, placenta-specific microRNAs. Prenat Diagn. 2015 January; 35 (1): 44-50. doi: 10.1002/pd.4479. Epub 2014 Sep. 17. PMID: 25125329.
74. Sadri M, Shu J, Kachman S D, Cui J, Zempleni J. Milk exosomes and miRNA cross the placenta and promote embryo survival in mice. Reproduction. 2020 October; 160 (4): 501-509. doi: 10.1530/REP-19-0521. PMID: 32621589.
75. Li H, Ouyang Y, Sadovsky E, Parks W T, Chu T, Sadovsky Y. Unique microRNA Signals in Plasma Exosomes from Pregnancies Complicated by Preeclampsia. Hypertension. 2020 March; 75 (3): 762-771. doi: 10.1161/HYPERTENSIONAHA.119.14081. Epub 2020 Jan. 27. PMID: 31983308; PMCID: PMC7076905.
76. Cleys E R, Halleran J L, Mcwhorter E, Hergenreder J, Enriquez V A, da Silveira J C, Bruemmer J E, Winger Q A, Bouma G J. Identification of microRNAs in exosomes isolated from serum and umbilical cord blood, as well as placentomes of gestational day 90 pregnant sheep. Mol Reprod Dev. 2014 November; 81 (11): 983-93. doi: 10.1002/mrd.22420. Epub 2014 Sep. 30. PMID: 25269776.
77. Rapado-González Ó, López-López R, López-Cedrún J L, Triana-Martínez G, Muinelo-Romay L, Suárez-Cunqueiro M M. Cell-Free microRNAs as Potential Oral Cancer Biomarkers: From Diagnosis to Therapy. Cells. 2019 Dec. 17; 8 (12): 1653. doi: 10.3390/cells8121653. PMID: 31861130; PMCID: PMC6952938.
78. Xiao J, Tao T, Yin Y, Zhao L, Yang L, Hu L. miR-144 may regulate the proliferation, migration and invasion of trophoblastic cells through targeting PTEN in preeclampsia. Biomed Pharmacother. 2017 October; 94:341-353. doi: 10.1016/j.biopha.2017.07.130. Epub 2017 Aug. 1. PMID: 28772212.
79. Hu S, Li J, Tong M, Li Q, Chen Y, Lu H, Wang Y, Min L. MicroRNA-144-3p may participate in the pathogenesis of preeclampsia by targeting Cox-2. Mol Med Rep. 2019 June; 19 (6): 4655-4662. doi: 10.3892/mmr.2019.10150. Epub 2019 Apr. 11. PMID: 31059003; PMCID: PMC6522833.
80. Zhou B, Zhang X, Li T, Xic R, Zhou J, Luo Y, Yang C. CircZDHHC20 represses the proliferation, migration and invasion in trophoblast cells by miR-144/GRHL2 axis. Cancer Cell Int. 2020 Jan. 13; 20:19. doi: 10.1186/s12935-020-1097-2. PMID: 31956297; PMCID: PMC6958575.
81. Williams K C, Renthal N E, Gerard R D, Mendelson C R. The microRNA (miR)-199a/214 cluster mediates opposing effects of progesterone and estrogen on uterine contractility during pregnancy and labor. Mol Endocrinol. 2012 November; 26 (11): 1857-67. doi: 10.1210/me.2012-1199. Epub 2012 Sep. 12. Erratum in: Mol Endocrinol. 2013 January; 27 (1): 188. PMID: 22973051; PMCID: PMC3487626.
82. Peng J, Jiang J, Wang H, Feng X, Dong X. miR-199a-3p suppresses cervical epithelial cell inflammation by inhibiting the HMGB1/TLR4/NF-κB pathway in preterm birth. Mol Med Rep. 2020 August; 22 (2): 926-938. doi: 10.3892/mmr.2020.11184. Epub 2020 May 22. PMID: 32468045; PMCID: PMC7339783.
83. Hu L, Ai J, Long H, Liu W, Wang X, Zuo Y, Li Y, Wu Q, Deng Y. Integrative microRNA and gene profiling data analysis reveals novel biomarkers and mechanisms for lung cancer. Oncotarget. 2016 Feb. 23; 7 (8): 8441-54. doi: 10.18632/oncotarget.7264. PMID: 26870998; PMCID: PMC4890978.
84. Pisano A, Griñan-Lison C, Farace C, Fiorito G, Fenu G, Jiménez G, Scognamillo F, Peña-Martin J, Naccarati A, Pröll J, Atzmüller S, Pardini B, Attene F, Ibba G, Solinas M G, Bernhard D, Marchal J A, Madeddu R. The Inhibitory Role of miR-486-5p on CSC Phenotype Has Diagnostic and Prognostic Potential in Colorectal Cancer. Cancers (Basel). 2020 Nov. 19; 12 (11): 3432. doi: 10.3390/cancers12113432. PMID: 33227890; PMCID: PMC7699298.
85. Tessema M, Yingling C M, Picchi M A, Wu G, Ryba T, Lin Y, Bungum A O, Edell E S, Spira A, Belinsky S A. ANK1 Methylation regulates expression of MicroRNA-486-5p and discriminates lung tumors by histology and smoking status. Cancer Lett. 2017 Dec. 1; 410:191-200. doi: 10.1016/j.canlet.2017.09.038. Epub 2017 Sep. 29. PMID: 28965852; PMCID: PMC5675764.
86. Feng L, Jing L, Han J, Wang G, Liu Y, Zhang X, Wang Y, Wang F, Ma H, Liu Y. MicroRNA 486-3p directly targets BIK and regulates apoptosis and invasion in colorectal cancer cells. Onco Targets Ther. 2018 Dec. 6; 11:8791-8801. doi: 10.2147/OTT.S180354. PMID: 30584337; PMCID: PMC6287550.
87. Huang X P, Hou J, Shen X Y, Huang C Y, Zhang X H, Xie Y A, Luo X L. MicroRNA-486-5p, which is downregulated in hepatocellular carcinoma, suppresses tumor growth by targeting PIK3R1. FEBS J. 2015 February; 282 (3): 579-94. doi: 10.1111/febs.13167. Epub 2014 Dec. 27. PMID: 25475121. J Clin Endocrinol Metab, Volume 102, Issue 9, 1 Sep. 2017, Pages 3182-3194
88. Li D B, Liu J L, Wang W, et al. Plasma Exosomal miRNA-122-5p and miR-300-3p as Potential Markers for Transient Ischaemic Attack in Rats. Front Aging Neurosci. 2018; 10:24. Published 2018 Feb. 6. doi: 10.3389/fnagi.2018.00024
89. Mong E F, Yang Y, Akat K M, Canfield J, VanWye J, Lockhart J, Tsibris J C M, Schatz F, Lockwood C J, Tuschl T, Kayisli U A, Totary-Jain H. Chromosome 19 microRNA cluster enhances cell reprogramming by inhibiting epithelial-to-mesenchymal transition. Sci Rep. 2020 Feb. 20; 10 (1): 3029. doi: 10.1038/s41598-020-59812-8. PMID: 32080251; PMCID: PMC7033247.
90. Gong H, Lu F, Zeng X, Bai Q. E2F transcription factor 1 (E2F1) enhances the proliferation, invasion and EMT of trophoblast cells by binding to Zinc Finger E-Box Binding Homeobox 1 (ZEB1). Bioengineered. 2022 February; 13 (2): 2360-2370. doi: 10.1080/21655979.2021.2023793. PMID: 35030974; PMCID: PMC8974018.
91. Manning B D, Toker A. AKT/PKB Signaling: Navigating the Network. Cell. 2017 Apr. 20; 169 (3): 381-405. doi: 10.1016/j.cell.2017.04.001. PMID: 28431241; PMCID: PMC5546324.
92. Woods L, Perez-Garcia V, Hemberger M. Regulation of Placental Development and Its Impact on Fetal Growth-New Insights From Mouse Models. Front Endocrinol (Lausanne). 2018 Sep. 27; 9:570. doi: 10.3389/fendo.2018.00570. PMID: 30319550; PMCID: PMC6170611.
93. Perry C H, Mullins N A, Sweileh R B A, Shendy N A M, Roberto P A, Broadhurst A L, Nelson H A, Miranda-Carboni G A, Abell A N. MAP3K4 promotes fetal and placental growth by controlling the receptor tyrosine kinases IGFIR/IR and Akt signaling pathway. J Biol Chem. 2022 September; 298 (9): 102310. doi: 10.1016/j.jbc.2022.102310. Epub 2022 Jul. 31. PMID: 35921893; PMCID: PMC9463538.
94. Luo S S, Ishibashi O, Ishikawa G, Ishikawa T, Katayama A, Mishima T, Takizawa T, Shigihara T, Goto T, Izumi A, Ohkuchi A, Matsubara S, Takeshita T, Takizawa T. Human villous trophoblasts express and secrete placenta-specific microRNAs into maternal circulation via exosomes. Biol Reprod. 2009 October; 81 (4): 717-29. doi: 10.1095/biolreprod. 108.075481. Epub 2009 Jun. 3. PMID: 19494253.

What is claimed is:

1. A non-invasive method for early diagnosis of a placental pathology comprising an abnormal formation or arrangement of a placenta in a uterus of a mammalian female subject during pregnancy, the method comprising
selectively purifying from plasma of maternal blood a population of small extracellular vesicles (small-EVs) expressing a placenta-specific surface biomarker; wherein the extracellular vesicles comprise micro-RNA cargo;
determining a cargo profile for the small EVs by extracting RNA from the purified population of small EVs; identifying and quantifying expression of small non-coding RNAs comprising one or more micro RNAs (miRNAs) encapsulated by the purified population of exosomes; and comparing the miRNA profile of the placenta specific EVs to the miRNA profile of a healthy control of the same approximate gestational age;
wherein the early diagnosis can lead to an improved maternal outcome.

2. The method according to claim 1, wherein the placental pathology includes placenta previa and placenta accrete spectrum (PAS).

3. The method according to claim 2, wherein the placenta accrete spectrum (PAS) comprises placenta accreta, placenta increta, and placenta percreta.

4. The method of claim 1, wherein the method comprises an initial ultrafiltration step, an ultracentrifugation step or both to provide a pooled heterogeneous population of biological particles.

5. The method of claim 1, wherein the purified population of small-EVs is at least 50% pure, at least 55% pure, at least 60% pure, at least 65% pure, at least 70% pure, at least 75% pure, at least 80% pure, at least 85% pure, at least 90% pure, at least 91%, 92%, 93%, 94%, 95%, 96%, 97%, 98% 99% or 100% pure.

6. The method of claim 1, wherein the purified population of small-EVs is homogeneous.

7. The method according to claim 1, wherein the selective purifying is by antibody capture of the placental EVs in the maternal plasma.

8. The method according to claim 7, wherein the antibody is a monoclonal antibody raised against a recombinant human PLAP and the placenta-specific biomarker comprises transmembrane placental alkaline phosphatase (PLAP) protein.

9. The method according to claim 8, wherein
a. the monoclonal antibody raised against the recombinant human PLAP is activated with a dibenzocyclo-octyl (DBCO)-ester;
b the DBCO-modified antibody is coupled to a DNA linker by click chemistry,
c. the antibody-DNA linker conjugates are bound to streptavidin coated well plates pretreated with RNAse A;
d. the purified population of placenta-specific small EVs are released from the streptavidin-coated well plates enzymatically by uracil glycosylase; and
e. the purified population of placenta-specific small-EVs is eluted from the monoclonal PLAP antibody complex by contacting the complex with free PLAP.

10. The method according to claim 1, wherein the method differentiates between small EVs of human women with the placental pathology placenta previa and human women with the placental pathology placenta percreta.

11. The method according to claim 10, wherein the method identifies 40 differentially expressed miRNAs, including miR-21 and, miR-191 and miR-223 with increased expression and miR-451 and miR-486 with decreased expression.

12. The method according to claim 10, wherein expression of has-miR-486, has-miR-151-3p, has-miR-378, has-miR-122, has-miR-199a-5p; and has-miR-340 are significantly differentially expressed between placenta previa and placenta percreta groups.

13. The method according to claim 10, wherein miRNAs in small-EVs purified from plasma of women with placenta percreta indicated an overall decrease in miRNA expression.

14. The method according to claim 10, wherein the top 14 miRNAs upregulated in placenta percreta play a role in regulation of genes involved in cell migration, cell proliferation and angiogenesis.

15. The method according to claim 14, wherein the genes include AKT1, IFGR1, TP53, PIK3C2A, ZEB1, and FOX01.

16. The method according to claim 10, wherein the top 9 down-regulated miRNAs in placenta percreta play a role in regulation of genes involved in cell proliferation, migration and sprouting angiogenesis.

17. The method according to claim 16, wherein the genes include KRAS, GSK3ß, and CCND1.
